(19) United States
(12) Patent Application Publication
(10) Pub. No.: US 2017/0240961 A1
(43) Pub. Date: Aug. 24, 2017

(54) AFFINITY TAG LABELED NUCLEOSIDES AND USES

(71) Applicant: Complete Genomics, Inc., Mountain View, CA (US)

(72) Inventors: Radoje Drmanac, Los Altos Hills, CA (US); Snezana Drmanac, Los Altos Hills, CA (US); Handong Li, San Jose, CA (US); Xun Xu, Sunnyvale, CA (US)

(73) Assignee: Complete Genomics, Inc., Mountain View, CA (US)

(21) Appl. No.: 15/440,920

(22) Filed: Feb. 23, 2017

Related U.S. Application Data
(60) Provisional application No. 62/298,818, filed on Feb. 23, 2016.

(51) Int. Cl. C12Q 1/68

(52) U.S. Cl. CPC C12Q 1/6869; C12Q 1/6876

(57) ABSTRACT

Nucleoside analogues and methods of using such nucleoside analogues for sequencing of nucleic acids are provided.

### REFERENCE TO RELATED APPLICATION

This application claims the priority benefit of U.S. provisional application 62/298,818, filed Feb. 23, 2016. The priority application is hereby incorporated herein in its entirety for all purposes.

### BACKGROUND OF THE INVENTION

The need for low cost, high-throughput, methods for nucleic acid sequencing and re-sequencing has led to the development “massively parallel sequencing” (MPS) technologies. Improvements in such sequencing methods are of great value in science, medicine and agriculture.

### BRIEF SUMMARY OF THE INVENTION

The present invention relates to novel nucleoside analogues and methods of their use for nucleic acid sequencing. In certain aspects, the invention relates to nucleoside analogues having a reversible 3′-O blocking group and an affinity tag linked to the nucleobase through a linker.

In a first aspect, the present invention provides a nucleoside analogue of the following formula:

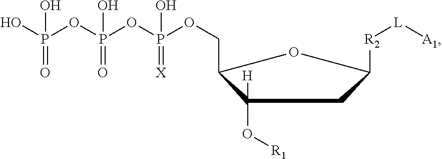

wherein R₁ is a reversible blocking group, e.g., selected from the group consisting of azidomethyl, nitrobenzyl, coumarinyl, nitronaphthalenyl, aminoxyl, and carbonyl; R₂ is a nucleobase; L is a linker; A₁ comprises a non-fluorescent affinity tag; X is selected from the group consisting of O and S; and the nucleoside analogue is a substrate for a DNA polymerase.

In some embodiments, L is a cleavable linker. In some embodiments, R₁ and L can be cleaved from the nucleoside analogue under the same conditions. In some embodiments, A₁ comprises a non-fluorescent affinity tag selected from the group consisting of nitrilotriacetic acid (NTA) and a peptide comprising at least six contiguous histidine (His) amino acids. In some embodiments, A₁ comprises a non-fluorescent affinity tag selected from the group consisting of biotin, vitamin D₃, a non-fluorescent small molecule antigen, and a peptide.

In some embodiments, the non-fluorescent small molecule antigen is selected from the group consisting of an amphetamine, a barbituate, a benzodiazepine, a cocaine metabolite, a cannabinoid, a cannabinoid metabolite, tetrahydrocannabinol, methadone, an opiate, propoxyphene, phencyclidine, digoxigenin, digoxin, and dinitrophenol (DNP). In some embodiments, the peptide antigen is selected from the group consisting of a His tag, a Myc tag, a Flag tag, an HA tag, a V5 tag, an AviTag, a calmodulin tag, an E tag, an S tag, an SBP tag, a Softag, a Strep tag, a TC tag, a VSV tag, an Xpress tag, glutathione, an isopeptag, and a SpyTag. In some embodiments, the nucleoside analogue comprises the following formula:

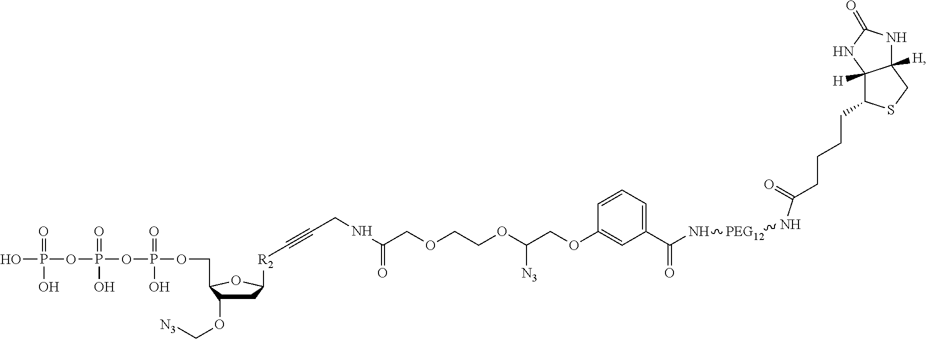

wherein R₂ is the nucleobase.

In some embodiments, the nucleobase is selected from the group consisting of a 7-substituted 7-deaza adenine analogue, a 7 substituted 7-deaza guanine analogue, a 5-substituted thymine, and a 5-substituted cytosine.

In a second aspect, the present invention provides a composition comprising: i) a nucleoside analogue of the following formula:

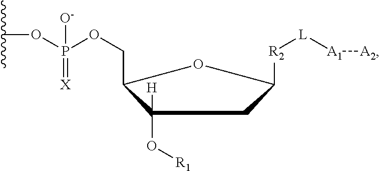

wherein R₁ is a reversible blocking group, e.g., selected from the group consisting of azidomethyl, nitrobenzyl, coumarinyl, nitronaphthalenyl, aminoxyl, and carbonyl; X is selected from the group consisting of O and S; R₂ is a nucleobase; L is a linker; A₁ comprises a fluorescent or non-fluorescent affinity tag; and A₂ comprises a detectably labeled affinity agent that forms a specific and non-covalent complex with A₁, wherein the nucleoside analogue is covalently linked via the 5′ phosphate or thiophosphate to an oligonucleotide. In some embodiments, A₁ comprises a fluorescent dye selected from the group consisting of a fluorone dye, a rhodamine dye, a cyanine dye, a coumarin dye, a phycoerythrin, and an allophycocyanine.

In a third aspect, the present invention provides a method of sequencing comprising: i) providing a reaction mixture comprising template nucleic acid, a primer, a polymerase, and a first nucleoside analogue of Formula VII:

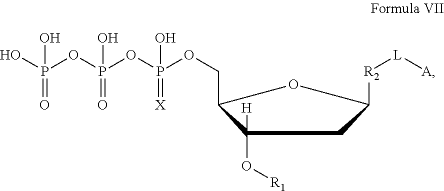

wherein R₁ is a reversible blocking group, e.g., selected from the group consisting of azidomethyl, nitrobenzyl, coumarinyl, nitronaphthalenyl, aminoxyl, and carbonyl; X is selected from the group consisting of O and S; R₂ is a nucleobase; L is a linker; and A comprises a non-fluorescent or fluorescent affinity tag; ii) extending the primer by incorporating the first nucleoside analogue with the polymerase; iii) contacting the incorporated first nucleoside analogue with a detectably labeled affinity agent that forms a specific and non-covalent complex with A of the incorporated first nucleoside analogue, thereby specifically labeling the incorporated first nucleoside analogue; and iv) detecting the specifically labeled incorporated first nucleoside analogue.

In some embodiments, the detectably labeled affinity agent is fluorescently labeled, and the detection comprises detecting a fluorescence emission from the fluorescently labeled affinity agent in complex with A of the incorporated first nucleoside analogue. In some embodiments, the method further comprises: v) cleaving the reversible blocking group R₁ of the incorporated first nucleoside analogue thereby removing the blocking group from the incorporated first nucleoside analogue; vi) cleaving the linker L thereby removing the non-fluorescent or fluorescent binding molecule A of the incorporated first nucleoside analogue, or quenching the label of the detectably labeled affinity agent in complex with A of the incorporated first nucleoside analogue; vii) providing a second nucleoside analogue of Formula VII and a polymerase; viii) extending the primer by incorporating the second nucleoside analogue with the polymerase; ix) contacting the incorporated second nucleoside analogue with a detectably labeled affinity agent that forms a specific and non-covalent complex with A of the incorporated second nucleoside analogue, thereby specifically labeling the incorporated second nucleoside analogue; and x) detecting the specifically labeled incorporated second nucleoside analogue.

In a fourth aspect, the present invention provides a method of sequencing comprising: i) providing a reaction mixture comprising template nucleic acid, a primer, a ligase, and an oligonucleotide comprising a 5′ portion and a 3′ portion comprising a first nucleoside analogue of Formula X:

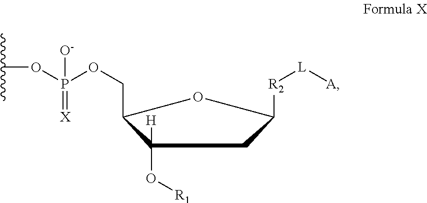

wherein R₁ is a reversible blocking group, e.g., selected from the group consisting of azidomethyl, nitrobenzyl, coumarinyl, nitronaphthalenyl, aminoxyl, and carbonyl; X is selected from the group consisting of O and S; R₂ is a nucleobase; L is a linker; A comprises a non-fluorescent or fluorescent affinity tag; and [symbol] denotes a 5′ phosphodiester bond between the nucleoside analogue of Formula X and the 5′ portion of the oligonucleotide; ii) hybridizing the oligonucleotide comprising the first nucleoside analogue of Formula X to the template nucleic acid at a position 3′ of, and adjacent to, the primer; iii) ligating the hybridized oligonucleotide to the adjacent primer with the ligase, thereby incorporating the first nucleoside analogue of Formula X into the primer; iv) contacting the incorporated first nucleoside analogue with a detectably labeled affinity agent that forms a specific and non-covalent complex with A of the incorporated first nucleoside analogue, thereby specifically labeling the incorporated first nucleoside analogue; and v) detecting the specifically labeled incorporated first nucleoside analogue.

In some embodiments, the detectably labeled affinity agent is fluorescently labeled, and the detection comprises detecting a fluorescence emission from the fluorescently labeled affinity agent in complex with A of the incorporated first nucleoside analogue. In some embodiments, the method further comprises: vi) cleaving the linker L thereby removing the non-fluorescent or fluorescent binding molecule A of the incorporated first nucleoside analogue, or quenching the label of the detectably labeled affinity agent in complex with A of the incorporated first nucleoside analogue; vii) cleaving the reversible blocking group R₁ of the hybridized oligonucleotide comprising the first nucleoside analogue of Formula X; viii) providing a second oligonucleotide, the oligonucleotide comprising a 5′ portion and a 3′ portion, wherein the 3′ portion comprises a second nucleoside analogue of Formula X, and a ligase; ix) hybridizing the second oligonucleotide comprising the second nucleoside analogue of Formula X to the template nucleic acid at a position 3′ of, and adjacent to, the primer; x) ligating the hybridized oligonucleotide to the adjacent primer with the ligase, thereby incorporating the second nucleoside analogue of Formula X into the primer; xi) contacting the incorporated second nucleoside analogue with a detectably labeled affinity agent that forms a specific and non-covalent complex with A of the incorporated second nucleoside analogue, thereby specifically labeling the incorporated second nucleoside analogue; and xii) detecting the specifically labeled incorporated second nucleoside analogue.

### BRIEF DESCRIPTION OF THE DRAWINGS

FIG. 1: shows an exemplary nucleoside analogue having a 3′ O reversible blocking group (R₁), and an affinity tag (A₁) covalently linked to a nucleobase (R₂).

FIG. 2: shows an exemplary nucleoside analogue after incorporation at the 5′ terminus of an extended primer and binding of detectably labeled affinity agent (A₂).

FIG. 3: shows an exemplary nucleoside analogue after incorporation into an extended primer. The 3′ O reversible blocking group (R₁) and a cleavable linker between the nucleobase and an affinity tag or fluorophore have been cleaved. The “ [symbol] ” indicates a 5′ phosphodiester bond with the remainder of the primer.

FIG. 4: shows an exemplary nucleoside analogue, wherein R₂ is the nucleobase, the reversible 3′-O blocking group is an azidomethyl, the linker comprises a cleavable azido alkyl, and the affinity tag is a biotin.

FIG. 5: shows Compound 12 (discussed in Example 1), an embodiment of the nucleoside analogue of FIG. 4, wherein the nucleobase R₂ is a guanosine analogue.

FIG. 6: shows results from HPLC analysis of Compound 12.

FIG. 7: shows results from ¹HNMR analysis of Compound 12.

FIG. 8: shows results from ³¹PNMR analysis of Compound 12.

FIG. 9: shows results from LC-MS analysis of Compound 12.

FIG. 10: shows results of a sequencing-by-synthesis run performed as described in Example 2.

FIG. 11 shows the surprising reduction in quenching afforded by use of affinity tag labeled nucleoside analogues in sequencing-by-synthesis methods as compared to conventional fluorescently labeled nucleoside analogues.

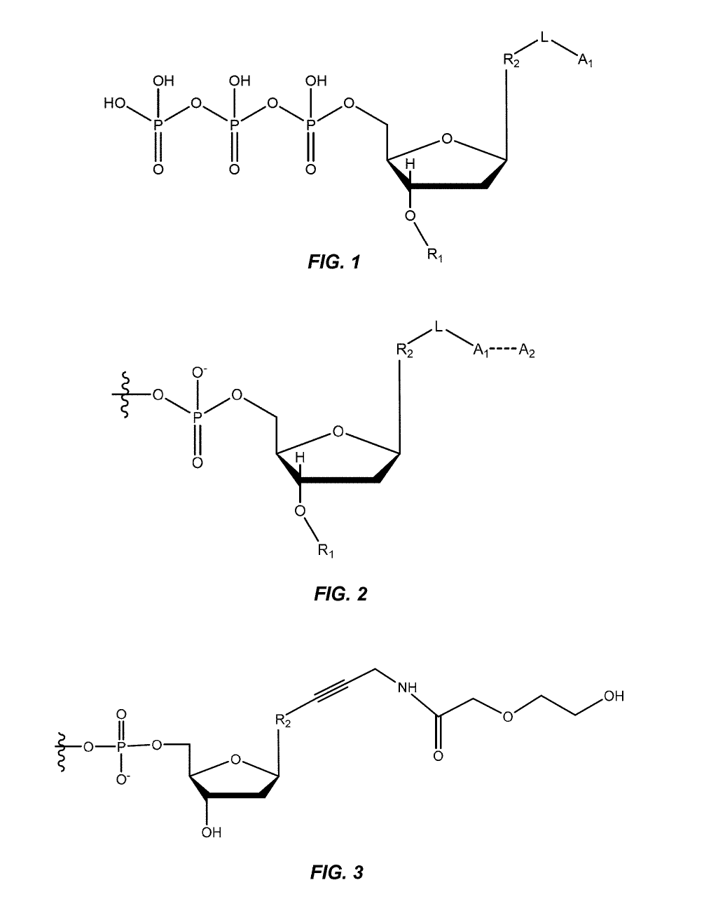

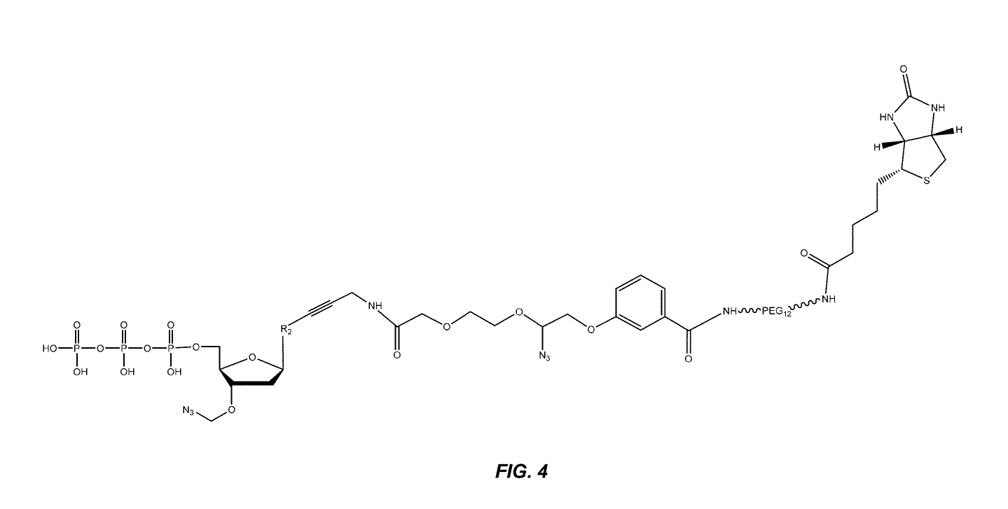

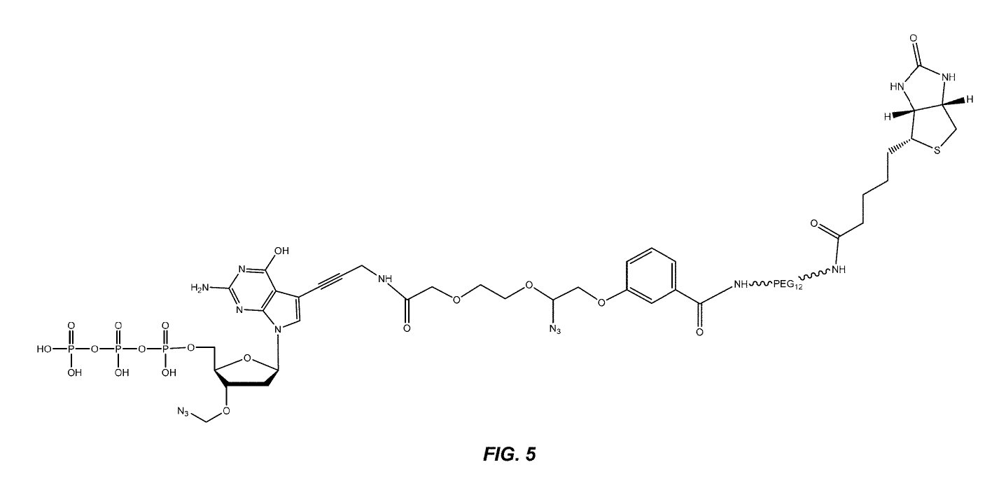

### DETAILED DESCRIPTION OF THE INVENTION

### I. Overview

In certain aspects, the present invention provides affinity tag labeled nucleoside analogues for nucleic acid sequencing. The affinity tag labeled nucleoside analogues can be reversibly blocked from polymerase- or ligase-mediated extension. The present invention also provides polynucleotides containing incorporated polymerase or ligase reaction products of such nucleoside analogues. The incorporated reaction products can be affinity tag labeled, or the affinity tag label can be cleaved. Similarly, the incorporated reaction products can be reversibly blocked or unblocked. In other aspects, the present invention provides methods of using such nucleoside analogues for nucleic acid sequencing.

In one aspect, the nucleoside analogue includes an affinity tag (e.g., biotin) attached via a cleavable linker to a reversibly blocked nucleobase (e.g., a 3′-O reversibly blocked nucleobase). The affinity tagged nucleoside analogue can be used in sequencing-by-synthesis or sequencing-by-ligation methods that include a step of detecting the affinity tag with a detectably labeled affinity agent. For example, the nucleoside analogue can be incorporated into a primer by a polymerase-mediated primer extension reaction. Alternatively, an oligonucleotide containing an incorporated analogue can be ligated to an anchor primer. The affinity tag in the extended primer or ligated oligonucleotide may then be bound by a detectably labeled affinity agent. The incorporated nucleoside or oligonucleotide containing the nucleoside can then be detected by detecting the bound affinity agent. The affinity tag labeled nucleoside analogues can be used in any compatible sequencing by synthesis or sequencing by ligation method known in the art.

### II. Definitions

As used herein, the term “complementary polynucleotide” refers to a polynucleotide complementary to a target nucleic acid. In one approach, the complementary polynucleotide is formed in a sequencing-by-synthesis reaction by sequential addition of nucleosides (e.g., naturally occurring nucleoside monophosphate molecules or analogs thereof) or groups of nucleosides to a primer using the target nucleic acid as a template.

As used herein, the term “nucleobase” refers to a nitrogenous base that can base-pair with a complementary nitrogenous base of a template nucleic acid. Exemplary nucleobases include adenine (A), cytosine (C), guanine (G), thymine (T), uracil (U), inosine (I) and derivatives of these. In one aspect, the nucleobase is a 7-deaza derivative of adenine, inosone, or guanine. In some cases, the 7-deaza adenine, inosine, and/or guanine derivatives are 7-substituted. In some cases, uracil, cytosine, thymine, and/or derivatives thereof are 5-substituted. Exemplary substitutions at the 7 position for adenine, inosine, or guanine derivatives or at the 5 position for cytosine, uracil, or thymine derivatives include an alkyne substitution (e.g., —C≡C—CH₂—NHR). In some cases, the 7-deaza derivative of guanine is a 7-alkyne substituted compound, such as found in compound 11:

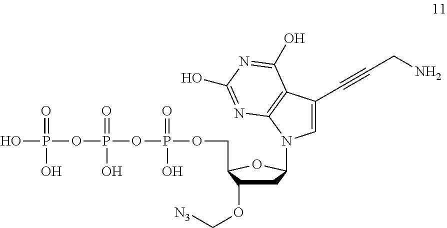

As used herein, the terms “affinity agent” and “affinity tag” refer to first and second members of a specific binding pair (SBP) or ligand-anti-ligand binding pair, where the members of the pair specifically bind to each other. For convenience, the term “affinity tag” is used to refer to the SBP member that is part of the nucleoside analog structure, and the term “affinity agent” is used to refer to the SBP member that specifically binds the affinity tag. The binding between the members of the binding pair is generally noncovalent, although a covalent (e.g., disulfide) linkage between binding pair members can also be used. In some cases, where a covalent linkage between binding pair members is used, the covalent linkage is reversible. For example, a covalent disulfide linkage can be cleaved with a reducing agent.

Binding between specific binding pairs results in the formation of a binding complex, sometimes referred to as a ligand/antiligand complex or simply as ligand/antiligand. Exemplary binding pairs include, but are not limited to: (a) a haptenic or antigenic compound in combination with a corresponding antibody, or binding portion or fragment thereof; (b) a nucleic acid aptamer and protein; (c) nonimmunological binding pairs (e.g., biotin-avidin, biotin-streptavidin, biotin-Neutravidin, biotin-Tamavidin, streptavidin binding peptide-streptavidin, glutathione-glutathione S-transferase); (d) hormone-hormone binding protein; (e) receptor-receptor agonist or antagonist; (f) lectin-carbohydrate; (g) enzyme-enzyme cofactor; (h) enzyme-enzyme inhibitor; (i) complementary oligonucleotide or polynucleotide pairs capable of forming nucleic acid duplexes; (j) thio (—S—) or thiol (—SH) containing binding member pairs capable of forming an intramolecular disulfide bond; and (k) complementary metal chelating groups and a metal (e.g., metal chelated by the binding pairs nitrilotriacetate (NTA) and a 6×-His tag). Specific binding pair members need not be limited to pairs of single molecules. For example, a single ligand can be bound by the coordinated action of two or more antiligands. Affinity agents can be detectably labeled.

In the context of the binding of an affinity agent to the affinity tag of a nucleoside analogue, the terms “specific binding,” “specifically binds,” and the like refer to the preferential association of an affinity agent with a nucleoside analogue bearing a particular target affinity tag in comparison to a nucleoside analogue base lacking the affinity tag or having an alternative affinity tag. Specific binding between an affinity agent and affinity tag generally means an affinity of at least 10⁻⁶ M⁻¹ (i.e., an affinity having a lower numerical value than 10⁻⁶ M⁻¹ as measured by the dissociation constant K_{d}). Affinities greater than 10⁻⁸ M⁻¹ are preferred. Specific binding can be determined using any assay for antibody binding known in the art, including Western Blot, enzyme-linked immunosorbent assay (ELISA), flow cytometry, immunohistochemistry, and detection of fluorescently labeled affinity agent bound to a nucleoside analogue bearing a target affinity tag in a sequencing reaction.

As used herein, the term “fluorescent dye” refers to a fluorophore (a chemical compound that absorbs light energy of a specific wavelength and re-emits light at a longer wavelength). Fluorescent dyes typically have a maximal molar extinction coefficient at a wavelength between about 300 nm to about 1,000 nm or of at least about 5,000, more preferably at least about 10,000, and most preferably at least about 50,000 cm⁻¹ M⁻¹, and a quantum yield of at least about 0.05, preferably at least about 0.1, more preferably at least about 0.5, and most preferably from about 0.1 to about 1. Exemplary fluorescent dyes include, without limitation, acridine dyes, cyanine dyes, fluorone dyes, oxazine dyes, phenanthridine dyes, and rhodamine dyes. Exemplary fluorescent dyes include, without limitation, fluorescein, FITC, Texas Red, ROX, Cy3, an Alexa Fluor dye (e.g., Alexa Fluor 647 or 488), an ATTO dye (e.g., ATTO 532 or 655), and Cy5. Exemplary fluorescent dyes can further include dyes that are used in, or compatible with, two- or four-channel ILLUMINA sequencing chemistries and workflows.

As used herein, the term “non-fluorescent affinity tag” refers to an affinity tag that is not a fluorescent dye (i.e., does not comprise a fluorophore). In various exemplary embodiments, “non-fluorescent affinity tag” refers an affinity tag that is not fluorescein or a fluorescein derivative, not a rhodamine fluorescent dye (e.g., Texas Red), not a cyanine fluorescent dye (e.g., Cy 2, Cy 3, Cy 3.5, Cy 5, Cy 5.5, Cy 7, Cy 7.5, and the like), not a boron-dipyrromethene fluorescent dye (e.g., BODIPY 493/503), not a fluorescent coumarin dye, not a phenoxazine fluorescent dye, not an acridine fluorescent dye, not an ALEXA FLUOR® fluorescent dye, not a DYLIGHT® fluorescent dye, not an ATTTO fluorescent dye, not a phycoerythrin fluorescent dye, or not an allophycocyanine fluorescent dye, or a combination of two or more or all thereof.

As used herein, the term “antigen” refers to a compound that can be specifically bound by an antibody. Some antigens are immunogens (see, Janeway, et al., Immunobiology, 5th Edition, 2001, Garland Publishing). Exemplary antigens used in the practice of the present invention, include polypeptides, small molecules, lipids, or nucleic acids (e.g., aptamers that are specifically bound by an antibody).

As used herein, the term “small molecule antigen” refers to a small molecule that can specifically bind an antibody. A “small molecule” in the context of a small molecule antigen refers to a molecule having a mass of less than about 1,000 Daltons (e.g., at least 50 Daltons and no more than about 1,000 Daltons). In some cases, the small molecule antigen is a small organic molecule. Exemplary small organic molecules include, but are not limited to, biotin or a derivative thereof (e.g., iminobiotin or biotin carboxylate), fluorescein or a derivative thereof (e.g., carboxyfluorescein or fluorescein isothiocyanate (FITC)), an amphetamine, a barbituate, a benzodiazepine, a cocaine metabolite, a marijuana metabolite, tetrahydrocannabinol (THC), methadone, an opiate, propoxyphene, phencyclidine (PCP), digoxigenin, digoxin, peptide antigens of less than about 1,000 Daltons, cholesterol or a derivative thereof, vitamin D₃, vitamin D₂, and steroid hormones (e.g., aldosterone, corticosterone, progesterone, dehydroepiandrosterone, 17β-Estradiol, etc.).

As used herein, the term “peptide antigen” refers to a primary sequence of amino acids that can specifically bind an antibody. Exemplary peptide antigens include, but are not limited to, a His tag, a Myc tag, a Flag tag, an HA tag, a V5 tag, an AviTag, a calmodulin tag, an E tag, an S tag, an SBP tag, a Softag, a Strep tag, a TC tag, a VSV tag, an Xpress tag, glutathione, an isopeptag, and a SpyTag.

As used herein, “antibody” refers to an immunoglobulin molecule (e.g., polyclonal and monoclonal antibodies), as well as genetically engineered forms such as chimeric antibodies (e.g., humanized murine antibodies), heteroconjugate antibodies (e.g., bispecific antibodies), recombinant single chain Fv fragments (scFv), and antigen binding forms of antibody fragments (e.g., Fab, F(ab)2, VH-VL Fab fragments).

The term “detectable label,” or “detection label,” as used herein, refers to any atom or molecule that can be used to provide a detectable and/or quantifiable signal. Suitable labels include radioisotopes, fluorophores, chromophores, mass labels, electron dense particles, magnetic particles, spin labels, molecules that emit chemiluminescence, electrochemically active molecules, enzymes, cofactors, and enzyme substrates. In some embodiments, the detection label is a molecule containing a charged group (e.g., a molecule containing a cationic group or a molecule containing an anionic group), a fluorescent molecule (e.g., a fluorescent dye), a fluorogenic molecule, or a metal. Optionally, the detection label is a fluorogenic label. A fluorogenic label can be any label that is capable of emitting light when in an unquenched form (e.g., when not quenched by another agent). The fluorescent moiety emits light energy (i.e., fluoresces) at a specific emission wavelength when excited by an appropriate excitation wavelength. When the fluorescent moiety and a quencher moiety are in close proximity, light energy emitted by the fluorescent moiety is absorbed by the quencher moiety. Optionally, the detection label is a fluorogenic dye. In some embodiments, the fluorogenic dye is a fluorescein, a rhodamine, a phenoxazine, an acridine, a coumarin, or a derivative thereof. In some embodiments, the fluorogenic dye is a carboxyfluorescein. Further examples of suitable fluorogenic dyes include the fluorogenic dyes commercially available under the ALEXA FLUOR product line (Life Technologies; Carlsbad, Calif.). Optionally, the label is a redoxgenic label. Optionally, the label is a reduction tag, a thio- or thiol-containing molecule, or a substituted or unsubstituted alkyl.

As used herein, the term “linker” refers to a chemical moiety that links a nucleoside analogue to an affinity tag and/or detectable label. Generally, linkers useful in the present invention can be up to 30 carbon atoms in length. Preferably, the linkers are 5-15 carbon atoms in length. The types of bonds between the linker and the nucleobase, the linker and the affinity tag, and/or the linker and the detectable label include, but are not limited to, amides, amines, esters, carbamates, ureas, thioethers, thiocarbamates, thiocarbonates, and thioureas, and other bonds known by those of ordinary skill in the art.

As used herein, the term “cleavable linker” refers to a chemical moiety that links a nucleoside analogue to an affinity tag and/or detectable label, and that can be cleaved to remove the affinity tag and/or detectable label from the nucleoside analogue. Cleavage can be performed using chemical or enzymatic methods.

### III. Compositions

### Nucleoside Analogues

Described herein are deoxyribose nucleoside analogues having a 3′ O reversible blocking group and a nucleobase. As used herein, the term “reversible blocking group” refers to a group that can be cleaved to provide a hydroxyl group at the 3′-position of the nucleoside analogue. The reversible blocking group can be cleavable by an enzyme, a chemical reaction, heat, and/or light. These 3′ O reversibly blocked deoxyribose nucleoside analogues can be used in a wide variety of nucleic acid sequencing methods, including but not limited to, sequencing by synthesis methods, and sequencing by ligation methods.

Such nucleoside analogues include but are not limited to those of Formula I:

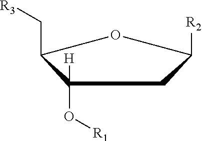

where R₁ is the 3′ O reversible blocking group, R₂ is, or includes, the nucleobase; and R₃ is a cleavable linking moiety comprising at least one (e.g., 1-10), at least two (e.g., 2-10), or at least three (e.g., 3-10), phosphates, or analogues thereof (e.g., a 5′-O-1-thiophosphate). The nucleoside analogue can be suitable as a substrate for an enzyme with DNA polymerase activity. In some cases, R₃ is a cleavable linking moiety comprising at least three (e.g., 3-10) phosphates, or analogues thereof, and the nucleoside analogue is suitable as a substrate for a DNA polymerase. R₂ can, e.g., be a nucleobase selected from adenine (A), cytosine (C), guanine (G), thymine (T), uracil (U), and derivatives of these. In some cases, the nucleoside analogue is a nucleoside triphosphate (i.e., R₃ consists of three consecutive phosphates).

In some embodiments, the reversible blocking group is an amino-containing blocking group (e.g., NH₂—). See, Hutter et. al, Nucleosides Nucleotides Nucleic Acids. 2010 November; 29(11), incorporated herein by reference in its entirety for all purposes, which describes exemplary amino-containing reversible blocking groups. In some embodiments, the reversible blocking group is an allyl-containing blocking group (e.g.,CH₂═CHCH₂—). In some embodiments, the reversible blocking group is an azido-containing blocking group (e.g., N₃—). In some embodiments, the reversible blocking group is azidomethyl (N₃CH₂—). In some embodiments, the reversible blocking group is an alkoxy-containing blocking group (e.g., CH₃CH₂O—). In some embodiments, the reversible blocking group contains a polyethylene glycol (PEG) moiety. In some embodiments, the reversible blocking group is a substituted or unsubstituted alkyl (i.e., a substituted or unsubstituted hydrocarbon). In some embodiments, the reversible blocking group is acyl. See, U.S. Pat. No. 6,232,465, incorporated herein by reference in its entirety for all purposes. In some embodiments, the reversible blocking group is or contains methoxymethyl. In some embodiments, the reversible blocking group is or contains aminoxyl (H₂NO—). In some embodiments, the reversible blocking group is or contains carbonyl (O═CH—).

In some embodiments, the reversible blocking group is nitrobenzyl (C₆H₄(NO₂)—CH₂—). In some embodiments, the reversible blocking group is coumarinyl (i.e., contains a coumarin moiety as depicted below, or a derivative thereof):

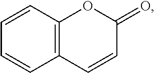

wherein, e.g., any one of the CH carbons of the coumarinyl reversible blocking group is covalently attached to the 3′ O of the nucleoside analogue.

In some embodiments, the reversible blocking group is nitronaphthalenyl (i.e., contains a nitronaphthalene moiety as depicted below, or a derivative thereof):

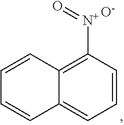

wherein, e.g., any one of the CH carbons of the nitronaphthalenyl reversible blocking group is covalently attached to the 3′ O of the nucleoside analogue.

The nucleoside analogues of Formula I can be labeled with a detectable label (e.g., a fluorescent label) at the R₂ and/or R₃ position. In an exemplary embodiment, the nucleoside analogues of Formula I are labeled with fluorescein, or Texas Red. Methods and compositions for labeling and detecting a nucleoside analogue with a detectable label such as a fluorophore, or a fluorophore/quencher pair, at the R₂ and/or R₃ position are described, e.g., in International Patent Publication Number WO 2016/065248, the content of which is hereby incorporated by reference in its entirety for all purposes.

In an exemplary embodiment, the nucleoside analogue of Formula I is a compound of Formula Ia:

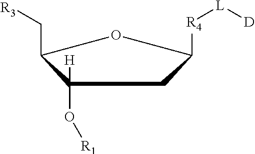

where R₁ is an 3′ O reversible blocking group, R₄ is a nucleobase; R₃ is a cleavable linking moiety containing three, or at least three (e.g., 3-10), phosphates, or analogues thereof; L is a linker; and D contains a detectable label (e.g., a fluorescent label). In some cases, R₃ contains at least one thiophosphate, such as a 5′-O-1-thiophosphate. R₄ can, e.g., be a nucleobase selected from adenine (A), cytosine (C), guanine (G), thymine (T), uracil (U), and derivatives of these. In some cases, R₃ is a cleavable linking moiety containing three phosphates, or analogues thereof. In some cases, the nucleoside analogue of Formula la is suitable as a substrate of a DNA polymerase, a ligase, or suitable as a substrate of a DNA polymerase and suitable as a substrate of a ligase.

In some cases, the deoxyribose nucleoside analogues include but are not limited to deoxyribose nucleoside triphosphate analogues of Formula II:

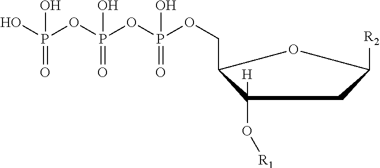

where R₁ is the 3′ O reversible blocking group, and R₂ is the nucleobase (e.g., an affinity tagged, fluorophore tagged, or untagged nucleobase).

In some cases, the deoxyribose nucleoside analogues include but are not limited to those of Formula III:

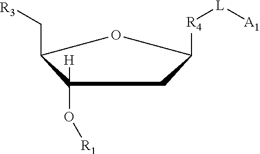

where R₁ is one of the foregoing 3′ O reversible blocking groups; R₂ is a nucleobase; R₃ is a cleavable linking moiety containing at least one (e.g., 1-10), at least two (e.g., 2-10), or at least three (e.g., 3-10), phosphates, or analogues thereof; L is a linker; and A₁ is a fluorescent or non-fluorescent affinity tag. In some cases, R₃ is a cleavable linking moiety comprising at least three (e.g., 3-10), phosphates, or analogues thereof. In some cases the nucleoside analogue is suitable as a substrate of a DNA polymerase, a ligase, or suitable as a substrate of a DNA polymerase and suitable as a substrate of a ligase. In some cases, R₃ comprises at least one thiophosphate, such as a 5′-O-1-thiophosphate.

The affinity tag can be, e.g., any of the affinity tags, ligands, or antiligands described herein. In some cases, A₁ is an antigen that can be specifically bound by an antibody. In some cases, A₁ is a non-fluorescent affinity tag. In some cases, A₁ is biotin. In some cases, A₁ is not biotin. In some embodiments, the affinity tag is an antibody, an amino acid, cholesterol, a lipid, FITC, Texas Red, an antigen, a peptide, a peptide antigen, a small molecule antigen, a steroid hormone, a drug, or a drug metabolite. Optionally, the affinity tag contains an oligonucleotide. In some cases, the affinity tag contains a thio (—S—) or a thiol (—SH) moiety.

In some cases, the affinity tag is or contains a metal chelating group (e.g., nitriloacetic acid (NTA), iminodiacetic acid (IDA), or a six-His peptide tag), optionally in complex with a metal (e.g., nickel, zinc, cobalt, copper, etc.). Such metal chelating groups, when in complex with a metal, can be detected with an detectably labeled affinity agent containing a complementary metal chelating group. Alternatively, a metal chelating group that is not in complex with a metal can be detected with a detectably labeled affinity agent containing or consisting of a metal or complementary metal-chelate. In some cases, chelate-metal-chelate complex or metal-chelate complex is detectable by detecting fluorescence of the metal-chelate or chelate-metal-chelate itself. For example, certain lanthanide chelates are suitable as a detectable label.

### Nucleoside Analogue Mixtures

The nucleoside analogues described herein can be provided or used in the form of a mixture. For example, the mixture can contain two, three, or four structurally different nucleoside analogues. The structurally different nucleoside analogues can differ at the nucleobase. For example, the mixture can contain four structurally different nucleoside analogues containing the four natural DNA nucleobases (i.e., adenine, cytosine, guanine, and thymine), or derivatives thereof. In some cases, each nucleoside analogue having a structurally different nucleobase can have a distinguishable detectable label or affinity tag. Alternatively, the mixture can contain four different nucleoside analogues but only three different detectable labels or affinity tags, wherein the fourth nucleoside analogue is unlabeled and/or untagged. Alternatively, the mixture can contain four structurally different nucleoside analogues that are used for two-color or two-channel sequencing, such that a first nucleoside analogue is labeled with a first affinity tag or detectable label, the second nucleoside analogue is labeled with a second affinity tag or detectable label, the third nucleoside analogue is labeled with the first and second affinity tag or detectable label (e.g., is a mixture of third nucleoside analogues labeled with the first affinity tag or label and third nucleotide analogues labeled with the second affinity tag or label), and the fourth nucleoside analogue is unlabeled and/or untagged.

### Linkers

A nucleoside analogue as described herein can be attached to a label (e.g., an affinity label and/or a detectable label) via a linker. Optionally, the linker used for attaching the nucleoside analogue to the label can be a cleavable linker. The linker can optionally be attached to the nucleobase of the nucleoside analogue. For example, the linker can be attached to the 5-position in a pyrimidine nucleobase or to the 7-position in a purine or deazapurine nucleobase. The linked can optionally be attached to a phosphate group located at the 5′-position of the nucleoside analogue.

Optionally, the linkers can be cleavable linkers. In nucleic acid sequencing and resequencing methods, the use of cleavable linkers between the nucleoside analogue and the label (e.g., the affinity label and/or the detectable label) allows the removal of the label after incorporation and detection of the nucleoside analogue. Optionally, the cleavable linkers are attached (e.g., covalently bonded) to the nucleoside analogue through the nucleobase of the nucleoside analogue. Optionally, the cleavable linkers are attached (e.g., covalently bonded) to the nucleoside analogue through a phosphate group at the 5′ position of the nucleoside analogue. The cleavable linkers as described herein can be cleaved to remove the label from the nucleoside analogue without otherwise altering the nucleoside analogue.

Cleavage can be performed using chemical or enzymatic methods. For example, cleavage can be performed by acid treatment, base treatment, oxidation, reduction, hydrolysis, or by photobleaching. Optionally, cleavage can be performed using phosphine-containing compounds or systems (e.g., phosphine-based transition metal catalysts or water-soluble phosphines). Optionally, cleavage can be performed using heat and/or light. The appropriate cleavage method depends on the nature of the linkage, which can be determined by those of ordinary skill in the art.

The cleavable linkers can include, for example, an azido group, an allyl group, a disulfide bond, an amide group, or an alkoxy group. Optionally, the cleavable linker contains at least one moiety that is present in the 3′-O reversible blocking group. For example, the cleavable linker can contain at least one of the following groups: allenyl, cyanoethyl, cyanoethenyl, formaldehyde oximyl, acrylaldehyde oximyl, propiolaldehyde oximyl, or cyanoethenaldehyde oximyl groups. Exemplary linkers for use as cleavable linkers in the nucleotide analogues described herein include the following moieties:

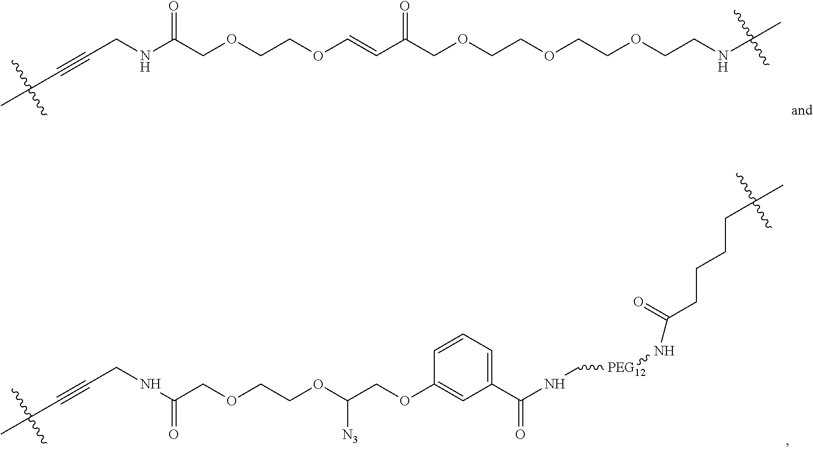

wherein the terminal “ [symbol] ” groups indicate where the moiety is connected to the remainder of the nucleoside analogue or the detectable label.

### Affinity Agents

Affinity agents can be used to detect the presence or absence of a nucleoside analogue, or a reaction product thereof, having a corresponding affinity tag. For example, the affinity agents can be used to detect incorporation of a nucleoside analogue having a corresponding affinity tag in a polynucleotide generated by a sequencing by synthesis or sequencing by ligation reaction.

In some embodiments, the affinity agent is a thio- or thiol-containing molecule, a protein, or a dendrimer. In some embodiments, the affinity agent is streptavidin, neutravidin, a tamavidin, glutathione S-transferase, thioredoxin, maltose binding protein, a lectin, or calmodulin binding protein. In some embodiments, the affinity agent is an antibody. In some embodiments, the affinity agent comprises an oligonucleotide complementary to an oligonucleotide affinity tag that is covalently linked to the nucleoside analogue. In some embodiments, the affinity agent comprises an oligonucleotide aptamer that specifically binds to an affinity tag (e.g., a peptide or protein affinity tag) that is covalently linked to the nucleoside analogue. In some embodiments, the affinity agent is a polynucleotide concatemer comprising multiple copies of a complementary affinity agent sequence or multiple copies of an aptamer affinity agent sequence. In some embodiments, the affinity agent contains a metal chelating group (e.g., a six-His tag, an NTA group, or an IDA group) capable of forming a ternary metal-chelate complex (chelate-metal-chelate) with a corresponding metal chelating group affinity tag covalently linked to the nucleoside analogue (e.g., a six-His tag, or an NTA, or IDA group).

One of ordinary skill in the art will recognize that an affinity agent and affinity tag binding pair can be interchanged. Thus the affinity agents described herein (e.g., unlabeled affinity agents) can be used as affinity tags (e.g., covalently linked to a nucleoside analogue). Moreover, the affinity tags described herein (e.g., detectably labeled affinity tags) can be used as affinity agents to detect the presence or absence of a nucleoside analogue.

### Detectable Labels

The affinity agents described herein can be detectably labeled. Detectably labeled affinity agents can be used to detect the presence or absence of a nucleoside analogue, or a reaction product thereof, having a corresponding affinity tag. Similarly, nucleobases or nucleoside analogues containing the nucleobase can be covalently linked to a detectable label (e.g., via a linker). The presence or absence of a detectably labeled nucleobase, nucleoside analogue, or reaction product thereof, can be detected to determine the sequence of a template nucleic acid. In some embodiments, the detectable label is a reporter molecule capable of generating a fluorescence signal. Exemplary reporter molecules are fluorescent organic dyes, which may be derivatized for attachment to an affinity agent, nucleobase, or nucleoside analogue.

There is a great deal of practical guidance available in the literature for selecting appropriate detectable labels for attachment to an affinity agent or nucleoside analogue, as exemplified by the following references: Grimm et al., 2013, “The chemistry of small-molecule fluorogenic probes,” Prog Mol Biol Transl Sci. 113:1-34, incorporated herein by reference, and Oushiki et al., 2012, “Near-infrared fluorescence probes for enzymes based on binding affinity modulation of squarylium dye scaffold,” Anal Chem. 84:4404-10; Medintz & Hildebrandt, editors, 2013, “FRET—Förster Resonance Energy Transfer: from theory to applications,” (John Wiley & Sons), and the like. The literature also includes references providing lists of fluorescent molecules, and their relevant optical properties for choosing fluorophores or reporter-quencher pairs, e.g., Haugland, Handbook of Fluorescent Probes and Research Chemicals (Molecular Probes, Eugene, 2005); and the like. Further, there is extensive guidance in the literature for derivatizing reporter molecules for covalent attachment via common reactive groups that can be added to a nucleoside, nucleobase, or affinity agent, as exemplified by the following references: Ullman et al., U.S. Pat. No. 3,996,345; Khanna et al., U.S. Pat. No. 4,351,760; and the like. Each of the aforementioned publications is incorporated herein by reference in its entirety for all purposes.

Exemplary reporter molecules may be selected from xanthene dyes, including fluoresceins, and rhodamine dyes. Many suitable forms of these compounds are widely available commercially with substituents on their phenyl moieties which can be used as the site for linking to an affinity agent. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate, and 2-p-toluidinyl-6-naphthalene sulfonate. Other dyes include 3-phenyl-7-isocyanatocoumarin; acridines, such as 9-isothiocyanatoacridine and acridine orange; N-(p-(2-benzoxazolyl)phenyl)maleimide; benzoxadiazoles; stilbenes; pyrenes; and the like.

In some embodiments, reporter molecules are selected from fluorescein and rhodamine dyes. These dyes and appropriate linking methodologies are described in many references, e.g., Khanna et al. (cited above); Marshall, Histochemical J., 7:299-303 (1975); Menchen et al., U.S. Pat. No. 5,188,934; Menchen et al., European Patent Application 87310256.0; and Bergot et al., International Application PCT/US90/05565. Fluorophores that can be used as detectable labels for affinity agents or nucleoside analogues include, but are not limited to, rhodamine, cyanine 3 (Cy 3), cyanine 5 (Cy 5), fluorescein, Vic™, Liz™, Tamra™, 5-Fam™, 6-Fam™, 6-HEX, CAL Fluor Green 520, CAL Fluor Gold 540, CAL Fluor Orange 560, CAL Fluor Red 590, CAL Fluor Red 610, CAL Fluor Red 615, CAL Fluor Red 635, and Texas Red (Molecular Probes).

By judicious choice of labels, analyses can be conducted in which the different labels are excited and/or detected at different wavelengths in a single reaction. See, e.g., Fluorescence Spectroscopy (Pence et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 1972; and Haugland, Handbook of Fluorescent Probes and Research Chemicals, Molecular Probes, Eugene (2005). In some embodiments, the presence or absence of nucleoside analogues having distinct affinity tags can be simultaneously and differentially detected with corresponding differentially labeled affinity agents. Such nucleoside analogue/affinity agent pairs can be used in two-, three-, or four-color sequencing.

Similarly, two-, three-, or four-color sequencing can be performed using differentially labeled nucleoside analogues having, e.g., a fluorescent, detectable label covalently linked (e.g., via a linker) to the nucleobase and/or 5′ phosphate. For example, nucleoside analogues comprising a reversible 3′ O blocking group and a detectable label covalently linked (e.g., via a linker) to the nucleobase or 5′ phosphate can be used in two-, three-, or four-color sequencing as described below.

### Labeled Oligonucleotides

Nucleoside analogues described herein can be used to sequence a template nucleic acid by a variety of methods. A variety of different oligonucleotides containing the nucleoside analogue, or a reaction product thereof, can be generated, depending on the sequencing method used and the nucleoside analogue employed. Examples of such oligonucleotides containing a nucleoside analogue of the present invention, or a reaction product thereof, are further described herein. Nucleoside analogues described herein, including those incorporated into an oligonucleotide, can also be useful in a variety of applications other than sequencing, as will be apparent to those of skill in the art. For example, nucleoside analogues described herein that include a fluorescent label covalently linked to a nucleobase can be used in a single nucleotide primer extension assay, such as described in Synvänen, AC, Nature Reviews Genetics 2, 930-942 (December 2001). As another example, nucleoside analogues described herein that include a fluorescent label covalently linked to an affinity tag can be used to hybridize to and isolate target nucleic acid fragments.

In one aspect, an oligonucleotide containing a nucleoside analogue, can be hybridized to a template nucleic acid adjacent to an anchor sequence. The oligonucleotide may be ligated to the anchor sequence and the oligonucleotide detected. Detection of the nucleoside analogue identifies the oligonucleotide and thus the sequence of bases complementary to the template nucleic acid, thereby providing the sequence of the template nucleic acid. A reversible blocking group of the incorporated nucleoside analogue, and optionally a detectable label or affinity tag, can be removed for further rounds of hybridization, ligation, and detection. In some embodiments, the nucleoside analogue comprises a fluorescent detectable label or affinity tag (e.g., linked to a nucleobase) and is incorporated into an oligonucleotide. In some cases, the nucleoside analogue is incorporated at the 3′ end of the oligonucleotide. Such 3′ end incorporation can be useful, e.g., for sequencing by ligation in the 5′ to 3′ direction.

Such oligonucleotides can comprise a nucleoside analogue of Formula IV:

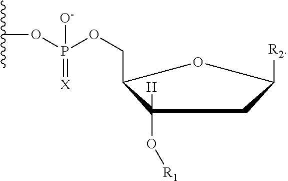

In some cases, R₁ is a reversible blocking group (e.g., selected from azidomethyl, nitrobenzyl, coumarinyl, nitronaphthalenyl, aminoxyl, and carbonyl); R₂ is or contains a nucleobase; and the nucleoside analogue is covalently linked via the 5′ phosphate to an oligonucleotide, wherein: [symbol] indicates the location of the 5′ phosphodiester bond to the oligonucleotide. X can be selected from O and S.

In an exemplary embodiment, R₂ contains or is a nucleobase, a linker, and a detectable label; or a nucleobase, a linker, and an affinity tag. In some cases, cleavage of the linker between nucleobase and detectable label or affinity tag can be performed under the same conditions as cleavage of the reversible blocking group. In some cases, the linker is not cleavable. In some cases, the linker is cleavable under orthogonal conditions relative to cleavage of the reversible blocking group.

In another aspect, a nucleoside analogue complementary to a template nucleic acid position and comprising a detectable label or an affinity tag (e.g., linked to a nucleobase) is incorporated into an oligonucleotide during a sequencing by synthesis reaction via a polymerase enzyme. Detection of the nucleoside analogue indicates the sequence of the base complementary to the template nucleic acid, thereby providing the sequence of the template nucleic acid. In some embodiments, the nucleoside analogue contains a fluorescent detectable label or affinity tag (e.g., linked to a nucleobase) and is incorporated into a oligonucleotide. Such oligonucleotides can include a nucleoside analogue of Formula IV, wherein R₁ is a reversible blocking group (e.g., selected from azidomethyl, nitrobenzyl, coumarinyl, nitronaphthalenyl, aminoxyl, and carbonyl); R₂ is or contains a nucleobase; and the nucleoside analogue is covalently linked via the 5′ phosphate to an oligonucleotide.

In an exemplary embodiment, R₂ includes a nucleobase, a linker, and a detectable label; or a nucleobase, a linker, and an affinity tag. In some cases, cleavage of the linker between nucleobase and detectable label or affinity tag can be performed under the same conditions as cleavage of the reversible blocking group. In some cases, the linker is not cleavable. In some cases, the linker is cleavable under orthogonal conditions relative to cleavage of the reversible blocking group.

Nucleoside analogues of Formula IV, wherein R₂ includes or is a nucleobase (e.g., a nucleobase that is not labeled with an affinity agent or detectable label) can also be utilized for sequencing by synthesis reaction schemes that do not rely on a specific step of detecting a distinguishable label. For example, pyrosequencing reaction schemes—in which nucleoside analogues of each of the four DNA nucleobases (A, G, C, and T) are delivered in succession and detected by a coupled assay for detection of pyrophosphate produced during incorporation of the nucleoside analogue by a polymerase—do not require a labeled or affinity tagged nucleobase.

Nucleoside analogues of Formula IV, wherein R₂ includes a nucleobase, a linker, and a detectable label can be covalently (e.g., via disulfide linkage) or non-covalently bound to a detectably labeled affinity agent. The presence or absence of the detectably labeled affinity agent can be determined to identify the sequence of the complementary base of the template nucleic acid in a sequencing by ligation or sequencing by synthesis reaction. Such oligonucleotides can contain a nucleoside analogue of Formula V:

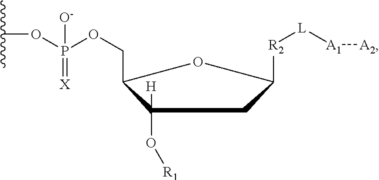

wherein R₁ is a reversible blocking group (e.g., selected from azidomethyl, nitrobenzyl, coumarinyl, nitronaphthalenyl, aminoxyl, and carbonyl); R₂ is a nucleobase; L is a linker; A₁ is or contains a fluorescent or non-fluorescent affinity tag; and A₂ is or includes a detectably labeled affinity agent that forms a specific complex with A₁. X can be selected from O and S.

In some cases, A₂ includes a detectably labeled affinity agent that forms a specific and non-covalent complex with A₁. In some cases, A₁ includes one or more thio (—S—) or thiol (—SH) groups, A₂ includes a detectably labeled affinity agent containing one or more thio (—S—) or thiol (—SH) groups, and A₂ forms a specific and covalent disulfide-mediated complex with A₁. In some cases, R₁ is a reversible blocking group.

In some cases, a 3′ O reversible blocking group can be cleaved prior to, or at the same time as, formation of a detection complex. In such cases, oligonucleotides produced in a sequencing by synthesis or sequencing by ligation reaction can contain a nucleoside analogue of Formula VI:

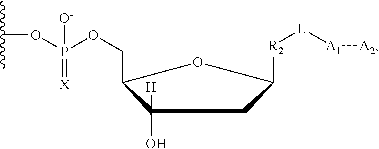

wherein R₂ is a nucleobase; X is selected from S and O; L is a linker; A₁ is or contains a fluorescent or non-fluorescent affinity tag; and A₂ is or contains a detectably labeled affinity agent that forms a specific complex (e.g., specific non-covalent complex, or specific, covalent, and disulfide mediated complex) with A₁.

### Reaction Mixtures

Nucleoside analogues and oligonucleotides containing such nucleoside analogues or reaction products thereof can be used as a component of a reaction mixture. For example, such components can be used in reaction mixtures for nucleic acid sequencing (e.g., sequencing by synthesis or by ligation). Exemplary reaction mixtures include, but are not limited to, those containing (a) template nucleic acid; (b) polymerase; (c) oligonucleotide primer; and (d) a 3′ O reversibly blocked nucleoside analogue, or a mixture of 3′ O reversibly blocked nucleoside analogues having structurally different nucleobases.

The reaction mixture can further optionally contain one or more of: ATP sulfurylase, luciferase, apyrase, adenosine 5′ phosphosulfate, and luciferin. In some cases, such a reaction mixture includes (a) template nucleic acid; (b) polymerase; (c) oligonucleotide primer; (d) a 3′ O reversibly blocked adenosine nucleoside analogue having an alpha thiophosphate; (e) ATP sulfurylase; (f) luciferase; (g) apyrase; (h) adenosine 5′ phosphosulfate; and (i) luciferin. In some cases, the adenosine nucleoside analogue having an alpha thiophosphate is an adenine nucleotide or derivative thereof that is not detectably labeled with a fluorophore or affinity tag, and contains a 3′ O reversible blocking group.

Alternatively, the reaction mixture can contain a 3′ O reversibly blocked nucleoside analogue, where the nucleobase is covalently linked to a linker, and the linker is linked to an affinity tag or detectable label. In some cases, the reaction mixture contains a mixture of nucleoside analogues having different nucleobases, where the nucleobases are covalently linked to a detectable and distinguishable label or affinity tag via a linker. In some cases, the reaction mixture further contains one or more detectably and distinguishably labeled affinity agents. Labeled nucleobases or labeled affinity agents can, e.g., be labeled with detectable fluorescent organic dyes, e.g., detectable and distinguishable fluorescent organic dyes.

### Template Nucleic Acids

In various embodiments, the template polynucleotide is DNA (e.g., cDNA, genomic DNA, or amplification products) or RNA. In various embodiments, the polynucleotide is double stranded or single stranded.

In some embodiments, the template nucleic acid is immobilized on a solid surface. In some embodiments, the template nucleic acid is immobilized on a substrate (e.g., a bead, flow cell, pad, channel in a microfluidic device and the like). The substrate may comprise silicon, glass, gold, a polymer, PDMS, and the like.

In some embodiments, the template nucleic acid is immobilized or contained within a droplet (optionally immobilized on a bead or other substrate within the droplet).

In some embodiments, the template nucleic acid is an immobilized DNA concatemer comprising multiple copies of a target sequence. In some embodiments, the template nucleic acid is represented as a DNA concatemer, such as a DNA nanoball (DNB) comprising multiple copies of a target sequence and an “adaptor sequence”. See International Patent Publication No. WO 2007/133831, the content of which is hereby incorporated by reference in its entirety for all purposes. In some embodiments the template is a single polynucleotide molecule. In some embodiments the template is present as a clonal population of template molecules (e.g., a clonal population produced by bridge amplification or Wildfire amplification).

It will be understood that the method is not limited to a particular form of template, and the template can be any template such as, for example, a DNA concatemer, a dendrimer, a clonal population of templates (e.g., as produced by bridge amplification or Wildfire amplification) or a single polynucleotide molecule. Thus, the specification should be read as if each reference to a template can alternatively refer to a concatemer template, a dendrimer, a clonal population of, e.g., short linear templates, a single molecule template (e.g., in a zero-mode waveguide), and templates in other forms.

Suitable template nucleic acids, including DNBs, clusters, polonys, and arrays or groups thereof, are further described in U.S. Pat. Nos. 8,440,397; 8,445,194; 8,133,719; 8,445,196; 8,445,197; 7,709,197; 12/335,168, 7,901,891; 7,960,104; 7,910,354; 7,910,302; 8,105,771; 7,910,304; 7,906,285; 8,278,039; 7,901,890; 7,897,344; 8,298,768; 8,415,099; 8,671,811; 7,115,400; 8,236,499, and U.S. Patent Publication Nos. 2015/0353926; 2010/0311602; 2014/0228223; and 2013/0338008, all of which are hereby incorporated by reference in their entireties for all purposes and particularly for all disclosure related to nucleic acid templates, concatemers and arrays according to the present invention.

### IV. Methods

### Cleavage of Blocking Groups or Linkers

Nucleoside analogues described herein can be 3′ O reversibly blocked. In some aspects, the blocking group provides for controlled incorporation of a single 3′ O reversibly blocked nucleoside analogue into a sequencing by synthesis primer, e.g., a sequencing by synthesis primer that has been extended in a previous cycle. Similarly, the blocking group provides for controlled incorporation of a single oligonucleotide containing a 3′ O reversibly blocked nucleoside analogue into an adjacent sequencing by ligation anchor primer or previously extended anchor primer. After incorporation and detection, the reversibly blocked nucleoside analogue, or an oligonucleotide containing such an analogue, can be treated to cleave the blocking group and allow further rounds of extension by polymerase or ligase.

The 3′ O reversible blocking group can be removed by enzymatic cleavage or chemical cleavage (e.g., hydrolysis). The conditions for removal can be selected by one of ordinary skill in the art based on the descriptions provided herein, the chemical identity of the blocking group to be cleaved, and nucleic acid chemistry principles known in the art. In some embodiments, the blocking group is removed by contacting the reversibly blocked nucleoside with a reducing agent such as dithiothreitol (DTT), or a phosphine reagent such as tris(2-carboxyethyl)phosphine (TCEP), tris(hydroxymethyl)phosphine (THP), or tris(hydroxypropyl)phosphine. In some cases, the blocking group is removed by washing the blocking group from the incorporated nucleotide analogue using a reducing agent such as a phosphine reagent. In some cases, the blocking group is photolabile, and the blocking group can be removed by application of, e.g., UV light. In some cases, the blocking group can be removed by contacting the nucleoside analogue with a transition metal catalyzed reaction using, e.g., an aqueous palladium (Pd) solution. In some cases, the blocking group can be removed by contacting the nucleoside analogue with an aqueous nitrite solution. Additionally, or alternatively, the blocking group can be removed by changing the pH of the solution or mixture containing the incorporated nucleotide analogue. For example, in some cases, the blocking group can be removed by contacting the nucleoside analogue with acid or a low pH (e.g., less than 4) buffered aqueous solution. As another example, in some cases, the blocking group can be removed by contacting the nucleoside analogue with base or a high pH (e.g., greater than 10) buffered aqueous solution.

3′ O reversible blocking groups that can be cleaved by a reducing agent, such as a phosphine, include, but are not limited to, azidomethyl. 3′ O reversible blocking groups that can be cleaved by UV light include, but are not limited to, nitrobenzyl. 3′ O reversible blocking groups that can be cleaved by contacting with an aqueous Pd solution include, but are not limited to, allyl. 3′ O reversible blocking groups that can be cleaved with acid include, but are not limited to, methoxymethyl. 3′ O reversible blocking groups that can be cleaved by contacting with an aqueous buffered (pH 5.5) solution of sodium nitrite include, but are not limited to, aminoalkoxyl.

In some aspects, the nucleoside analogue contains a 3′ O reversible blocking group and a linker between the nucleobase and an affinity tag or detectable label. In such cases, it can be advantageous to cleave the linker and thereby remove the affinity tag or detectable label from the nucleobase. In some embodiments, the cleavage is performed under the same conditions as the cleavage of the 3′ O reversible blocking group, providing simultaneous cleavage of blocking group and label or affinity tag. For example, 3′ O azidomethyl can be cleaved with a phosphine reagent under the same conditions as a linker containing an azidomethylether (N₃—CHR₁—OR₂) or a disulfide (S—S) within the linker element. Simultaneous cleavage can be used to reduce the number of processing steps required during multiple rounds of nucleoside incorporation, detection, and cleavage. Alternatively, the cleavage can be performed under orthogonal conditions. Orthogonal cleavage can be used to control the relative order in which the nucleoside analogue is unblocked and label or labeled affinity agent is removed. The cleavage can be performed according to any of the methods described above for cleavable linkers, including chemical or enzymatic methods. For example, cleavage can be performed by acid treatment, base treatment, oxidation, reduction, hydrolysis, or by photobleaching. Optionally, cleavage can be performed using phosphine-containing compounds or systems (e.g., phosphine-based transition metal catalysts or water-soluble phosphines). In some cases, the label or labeled affinity agent is removed by linker cleavage prior to cleavage of reversible blocking group. In some cases, the label or labeled affinity agent is removed by linker cleavage before cleavage of reversible blocking group.

Detection is generally performed prior to linker cleavage, if linker cleavage is employed. However, detection can be performed before or after reversible blocking group cleavage. Moreover, in some embodiments, although the nucleoside analogue can contain a group comprising a nucleobase that is covalently linked to a cleavable linker (which in turn is covalently linked to a detectable label or affinity tag), cleavage of the linker is not universally employed or required for performing additional cycles of sequencing-by-synthesis or sequencing-by-ligation. For example, detectable label covalently linked to the linker or detectably labeled affinity agent can be quenched in lieu of, or in addition to, linker cleavage. Additionally, or alternatively, detectably labeled affinity agent can be stripped from the affinity tag. For example, a detectably labeled antibody or other affinity agent, can be stripped from an affinity tag with a low pH (e.g., 100 mM glycine pH 2.8) or high pH (e.g., 100 mM glycine pH 10), high salt, or chaotropic stripping buffer.

### Sequencing

The nucleoside analogues described herein can be used in a variety of sequencing methods. For example, the analogues can be used in no-label, 2-label, 3-label, or 4-label sequencing methods. Exemplary no-label sequencing methods include, but are not limited to, methods in which nucleoside analogues having different nucleobases (e.g., A, C, G, T) are delivered in succession and incorporation is detected by detecting the presence or absence of the same signal or label for each different nucleobase. Thus, no-label methods are sometimes known as one-label, or one-color methods because the detection signal and/or label is the same for all nucleobases. For example, incorporation of a nucleoside into a primer by DNA polymerase mediated template directed polymerization can be detected by detecting a pyrophosphate cleaved from the nucleoside pyrophosphate. Pyrophosphate can be detected using a coupled assay in which ATP sulfurylase converts pyrophosphate to ATP, in the presence of adenosine 5′ phosphosulfate, which in turn acts as a substrate for luciferase-mediated conversion of luciferin to oxyluciferin, generating visible light in amounts proportional to ATP generation.

In an alternative no-label system, an inducer that is released by polymerase-mediated cleavage between alpha and beta phosphate of a nucleoside analogue, and optionally further processed by a second enzyme such as a phosphatase or sulfurylase, then activates a quenched dye on a capture element. This system, methods, and compositions for performing the methods, are further described, e.g., in International Patent Publication Number WO 2016/065248, the contents of which are hereby incorporated by reference in their entirety for all purposes.

One of skill in the art will recognize that, although a nucleoside analogue containing a nucleobase-linker moiety attached to a detectable label or affinity agent group is not required for such no-label methods, such nucleoside analogues are compatible with no-label methods in general. As such, no-label sequencing methods can employ any one of the following nucleoside analogues, or mixtures thereof.

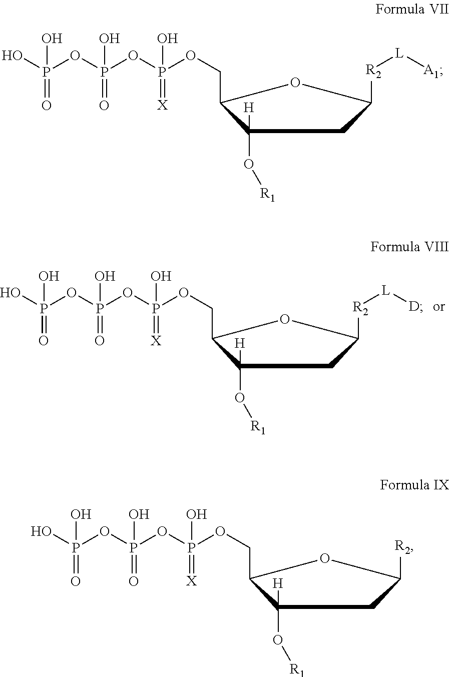

wherein X is selected from O and S; R₁ is or includes a 3′ O reversible blocking group; R₂ is or includes a nucleobase; L is or includes a linker (e.g., a cleavable linker); D is or includes a detectable label (e.g., detectable fluorescent label); and A₁ is or includes an affinity tag.

Alternatively, 2-label sequencing can be performed with the nucleoside analogues described herein, using two distinguishable signals in a combinatorial fashion to detect incorporation of four different nucleobases. Exemplary 2-label systems, methods, and compositions include, without limitation, those described in U.S. Pat. No. 8,617,811, the contents of which are hereby incorporated by reference in the entirety for all purposes and particularly for disclosure related to 2-label sequencing. Briefly, in 2-label sequencing, incorporation of a first nucleobase (e.g., A) is detected by detecting the presence of a first label; and, incorporation of a second nucleobase (e.g., C) is detected by detecting the presence of a second label. Incorporation of a third nucleobase (e.g., T) is detected by detecting the presence of both the first and second label attached to the third nucleobase; and, incorporation of a fourth unlabeled nucleobase (e.g., G) is detected by detecting the absence of both first and second labels. The labels of the nucleoside analogues utilized in a 2-label sequencing method can be attached to affinity agents specifically bound to affinity tags linked (e.g., cleavably linked) to a nucleobase, or directly attached to via a covalent linker (e.g., cleavable linker) to the nucleobase.

Similarly, 3- and 4-label sequencing can be performed with the nucleoside analogues described herein, using three or four distinguishable signals to detect incorporation of four different nucleobases. For example, 3-label sequencing can employ a first nucleobase labeled with a first label, a second nucleobase labeled with a second label, a third nucleobase labeled with a third label, and a fourth nucleobase that is either not labeled or labeled with a combination of first and second, first and third, or second and third labels. The labels of the nucleoside analogues utilized in a 3-label sequencing method can be attached to affinity agents specifically bound to affinity tags that are in turn linked (e.g., cleavably linked) to a nucleobase, or directly attached to via a covalent linker (e.g., cleavable linker) to the nucleobase. Similarly, 4-label sequencing can employ a first nucleobase labeled with a first label, a second nucleobase labeled with a second label, a third nucleobase labeled with a third label, and a fourth nucleobase labeled with a fourth label. The labels of the nucleoside analogues utilized in a 4-label sequencing method can be attached to affinity agents specifically bound to affinity tags that are in turn linked (e.g., cleavably linked) to a nucleobase, or directly attached via a covalent linker (e.g., cleavable linker) to the nucleobase.

Such 2-, 3-, and 4-label, also referred to as 2-, 3-, and 4-color, sequencing methods can be used in both sequencing by synthesis and sequencing by ligation. For example, nucleoside analogues of Formulas VII and VIII can be utilized for sequencing by synthesis in a 2-, 3-, or 4-label method. Similarly, oligonucleotides containing nucleoside analogues of the following formulas can be used for sequencing by ligation in a 2-, 3-, or 4-label method:

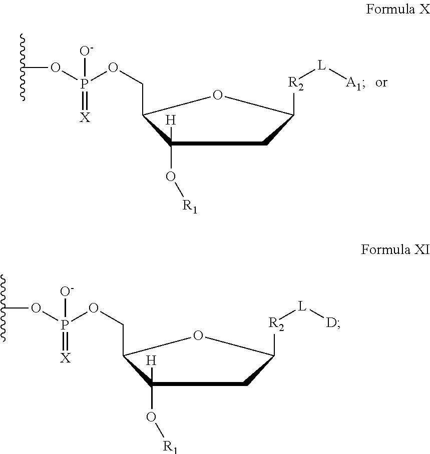

wherein X is selected from S and O; R₁ is a 3′-O reversible blocking group; R₂ is or includes a nucleobase; L is or includes a linker; D is or includes a detectable label (e.g., a detectable fluorescent label); and A₁ is or includes an affinity tag.

Various sequencing by synthesis and sequencing by ligation methods can be used with the nucleoside analogues of the present invention. In some aspects, the sequencing by synthesis methods can be selected from those described in U.S. Pat. Nos. 6,210,891; 6,828,100, 6,833,246; 6,911,345; 6,969,488; 6,897,023; 6,833,246; and 6,787,308; Patent Publication Nos. 2003/0064398; and 2003/0022207; Margulies et al., 2005, Nature 437:376-380; Ronaghi et al., 1996, Anal. Biochem. 242:84-89; Constans, A, 2003, The Scientist 17(13):36; and Bentley et al., 2008, Nature 456(7218): 53-59. In some aspects, sequencing by ligation methods can be selected from those described in International Patent Publication WO 1999/019341; WO 2005/082098; WO 2006/073504; and Shendure et al., 2005, Science, 309: 1728-1739. In an exemplary embodiment, the sequencing by synthesis or sequencing by ligation is performed using one or more nucleoside analogues described herein with a method described in International Patent Publication Number WO 2016/133764, the contents of which is hereby incorporated by reference in its entirety for all purposes, and particularly for the template preparation and sequencing methods and compositions described therein.

For example, a DNA strand for sequencing can be produced by a) providing a template DNA polynucleotide containing a first target DNA sequence interposed between a first adaptor 3′ to the first target DNA sequence and a second adaptor 5′ to the first target DNA sequence, and optionally comprising a third adaptor 3′ to the first adaptor and a second target DNA sequence interposed between the first adaptor and the third adaptor, wherein the template DNA polynucleotide is immobilized on a substrate; b) combining a first primer with the immobilized template DNA polynucleotide, and hybridizing the first primer to a first primer binding sequence in the first adaptor, wherein the first primer is not immobilized on the substrate when it is combined with the immobilized template DNA polynucleotide; c) extending the first primer using a first DNA polymerase to generate a second strand, wherein the second strand comprises a sequence complementary to the first target DNA sequence and a sequence complementary to at least part of the second adaptor; d) combining a second primer with the immobilized template DNA polynucleotide, hybridizing a second primer to a second primer binding sequence, wherein the second primer binding sequence is 3′ to the first primer binding sequence, wherein the second primer is not immobilized on the substrate when it is combined with the immobilized template DNA polynucleotide; e) extending the second primer using a DNA polymerase having strand-displacement activity to generate a third strand, wherein extending the second primer to generate the third strand partially displaces the second strand, thereby producing a partially hybridized second strand having: (i) a hybridized portion that is hybridized to the template DNA polynucleotide; and (ii) an unhybridized overhang portion that contains a sequence that is complementary to the first target DNA sequence and a sequence that is complementary to at least part of the second adaptor, wherein the unhybridized portion is 5′ in the second strand to the hybridized portion.

The prepared template can be then sequenced by, e.g., hybridizing a sequencing oligonucleotide to the sequence in the third strand that is complementary to at least part of the second adaptor. The sequencing oligonucleotide can be an anchor primer for hybridizing to template nucleic acid and ligating to an adjacent hybridized oligonucleotide containing a nucleoside analogue described herein. Thus, the method can be used for sequencing by ligation. Alternatively, the sequencing oligonucleotide can be a polymerase primer that is extended by incorporating a nucleoside analogue described herein. Thus, the method can be used for sequencing by synthesis.

### EXAMPLES

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope

### Example 1

### Synthesis of Nucleoside Analogues

### Synthesis of Linker-PEG-Biotin:

General Procedure for the Preparation of Compound 2 (3-([1,3]-dioxolan-2-ylmethoxy)-benzoic acid ethyl ester)

A mixture of 2-bromomethyl-1,3-dioxolane (8.3 mL, 80 mmol), ethyl-3-hydroxy-benzoate (3.32 g, 20 mmol), potassium carbonate (5.53 g, 40 mmol) and sodium iodide (1.2 g, 8 mmol) in dimethylformamide (DMF; 8 mL) was heated at 120° C. for 17 hours. The reaction was cooled to room temperature and all the solvents were evaporated under reduced pressure. The residue was partitioned between dichloromethane (DCM; 50 mL) and water (50 mL). The DCM layer was separated and the aqueous layer was back-extracted with DCM (2×100 mL). All the DCM extracts were combined, dried over sodium sulfate and evaporated under reduced pressure. The residue was purified by column chromatography using pentane (PE) and ethyl acetate (EA) (PE/EA ratio of 10/1 to 5/1) to give compound 2 (3.5 g, 69%), Rf=0.3 (PE/EA, 5/1).

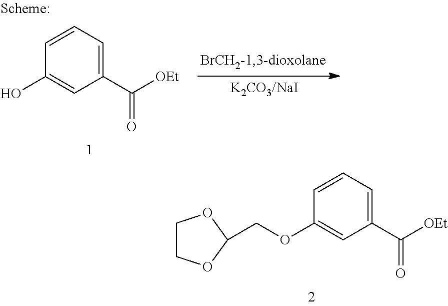 [SMILES: CCOC(=O)c1cccc(O)c1 ; CCOC(=O)c1cccc(OCC2OCCO2)c1]

General Procedure for the Preparation of Compound 3 (3-[2-azido-2-(2-hydroxy-ethoxy)-ethoxy]-benzoic acid ethyl ester)

To a mixture of 3-([1,3]-dioxolan-2-ylmethoxy)-benzoic acid ethyl ester (3.5 g, 13.9 mmol) and azidotrimethylsilane (2.35 mL, 15.3 mmol) was added tin (IV) chloride (1.2 mL) at room temperature under nitrogen. After 2 hours, 2% aqueous methanol (20 mL) was added to the reaction mixture and the reaction was stirred at room temperature for 30 minutes. All the solvents were evaporated under reduced pressure. The residue was co-evaporated with ethanol. The residue was purified by a column chromatography (PE/EA, 10/1 to 5/1). The title compound was obtained as a colorless oil (3.2 g, 78%), Rf=0.5 (PE/EA, 2/1).

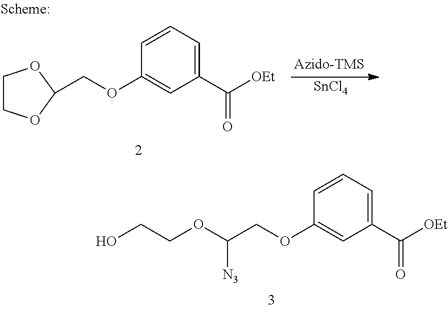

General Procedure for the Preparation of Compound 4 (3-[2-azido-2-(2-hydroxy-ethoxy)-ethoxy]-benzoic acid)

A mixture of 3-[2-azido-2-(2-hydroxy-ethoxy)-ethoxy]-benzoic acid ethyl ester (3.2 g, 10.8 mmol) was stirred with 4 M aqueous sodium hydroxide (27 mL) and ethanol (30 mL) at room temperature. After stirring for 3 hours, the solvents were removed under reduced pressure and the residue was dissolved in water (25 mL). The solution was acidified with 1 N HCl to pH 2 and extracted with DCM (3×25 mL). The DCM extracts were combined, dried over sodium sulfate and evaporated under reduced pressure. The title compound was obtained as a colorless solid (2.5 g, 95%), which was used for next step without further purification.

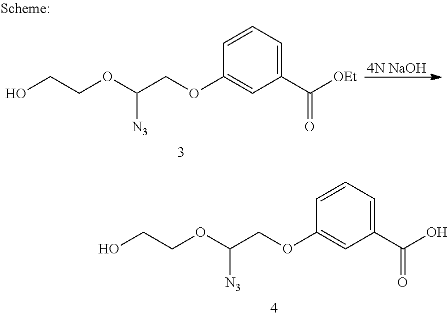

General Procedure for the Preparation of Compound 5 (3-[2-azido-2-(2-ethoxycarbonylmethoxy-ethoxy)-ethoxy]-benzoic acid)

To a solution of 3-[2-azido-2-(2-hydroxy-ethoxy)-ethoxy]-benzoic acid (1.0 g, 3.75 mmol) in dry tetrahydrofuran (THF; 10 mL) was added NaH (60% dispersion, 0.45 g, 11.2 mmol) at 0° C. After 10 minutes, ethyl-2-bromoacetate (8.0 mL) was added. The reaction was warmed up to room temperature and stirred overnight. The reaction was quenched by pouring into ice-cold water (50 mL). The mixture was extracted with DCM (2×25 mL) and the organic extracts were discarded. The aqueous layer was acidified to pH 2 with 1 N HCl, and extracted with DCM (3×350 mL). These DCM extracts were combined, dried over sodium sulfate and evaporated under reduced pressure. The residue was purified by column chromatography (PE/EA, 1/1 to 1/5) to obtain the compound 5 (0.6 g, 46%) as an oil.
LC-MS: 352 (M-11
Rf=0.7 (PE/EA, 1/5)

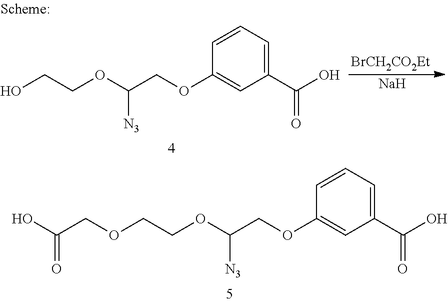

### General Procedure for the Preparation of Compound 6

To a mixture of PEG12-diamine (18.7 mmol, 1 eq), pyridine (2.3 mL, 29.4 mmol, 1.6 eq) in DCM (30 mL) cooled in an ice-bath was added 2,2,2-trifluoroacetic anhydride (4.0 mL, 28.4 mmol, 1.5 eq). The reaction mixture was stirred at room temperature overnight. The mixture was concentrated. The resultant residue was dissolved in minimum amount of dry DCM. The product was crashed out into dry hexane. The filter cake was dried to provide compound 6 (73%) as a yellow solid, which was used for next step without further purification.

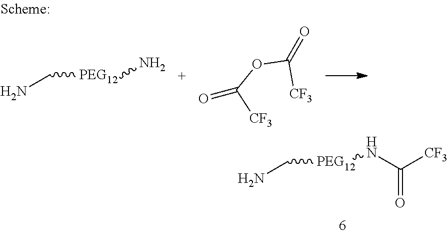

### General Procedure for the Preparation of Compound 7

To a mixture of 3-[2-azido-2-(2-ethoxycarbonylmethoxy-ethoxy)-ethoxy]-benzoic acid (0.72 g, 2.05 mmol), HATU (i.e., 1-[Bis(dimethylamino)methylene]-1H-1,2,3-triazolo[4,5-b]pyridinium 3-oxid hexafluorophosphate)) (0.94 g, 2.45 mmol), DIPEA (i.e., N,N-diisopropylethylamine) (0.8 g, 6.15 mmol) in THF (10 mL) was added amine compound 6 (2.46 mmol). The reaction mixture was stirred at room temperature for 1 hour. The mixture was extracted with EA/PE (1/1) (25 mL) and water (10 mL). The organic layer was separated and washed with 10% HCl, dried over sodium sulfate and concentrated under reduced pressure. The residue was purified by column chromatography (PE/EA, 1/1 to 1/5) to give the compound 7 (63%).

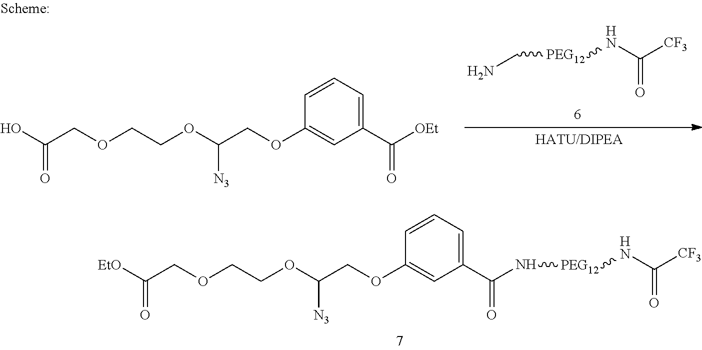

### General Procedure for the Preparation of Compound 8

Compound 7 (1.28 mmol) was stirred with 4N aqueous sodium hydroxide (3.2 mL, 12.8 mmol) and ethanol (4 mL) at room temperature. After 2 hours, all the solvents were removed under reduced pressure and the residue was dissolved in water (15 mL). The solution was extracted with DCM (2×20 mL). The DCM extracts were discarded and the aqueous layer was acidified with 1 N HCl to pH 2. Then the solution was extracted again with DCM (3×15 mL). The DCM extracts were discarded and the aqueous layer was neutralized with 1 N NaOH to pH 8 and then evaporated under reduced pressure to dryness. The white solids were triturated with DCM/methanol (MeOH) (v/v; 1:1, 2×25 mL). All the solids were filtered off and the filtrates were combined and evaporated under reduced pressure to give a gum. The gum was added in 10% MeOH in DCM (15 mL) and the insoluble, white solids were filtered off. The filtrates were evaporated under reduced pressure to give the mono-sodium salt of the title compound 8 (84%) as white powder.

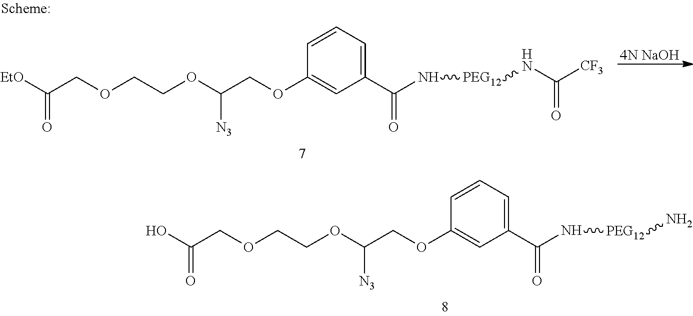

### General Procedure for the Preparation of Compound 9

To a solution of biotin-OSu (0.014 mmol) in DMF (1.0 mL) at 0° C. was added compound 8 (20.2 mg, 0.055 mmol) and saturated sodium bicarbonate (0.2 mL). The mixture was slowly warmed to room temperature and stirred overnight. LC-MS indicated the reaction was complete. The mixture was purified by preparative high performance liquid chromatography (prep-HPLC) to give pure 9 (53%).

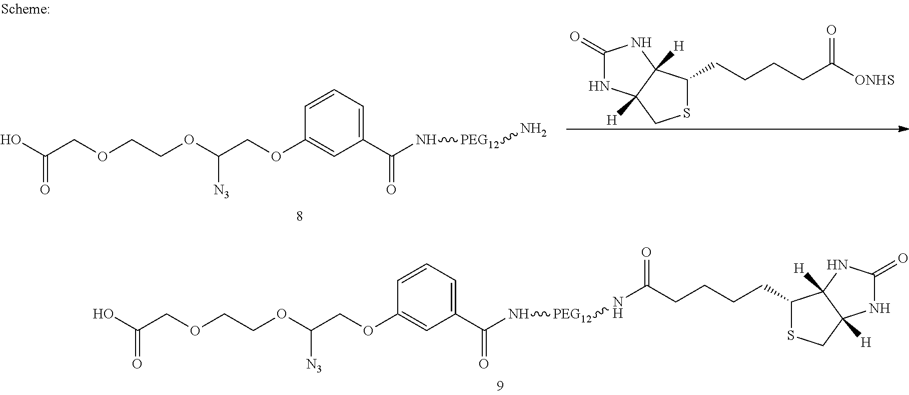

### General Procedure for the Preparation of Compound 10

To a solution of compound 9 (4.9 mg, 5 μmol) in DMF (0.5 mL) was added TSTU (2.3 mg, 7.5 μmol) and triethylamine (1.4 uL, 10 μmol). After 2 hours, LCMS indicated the complete formation of OSu compound 10.

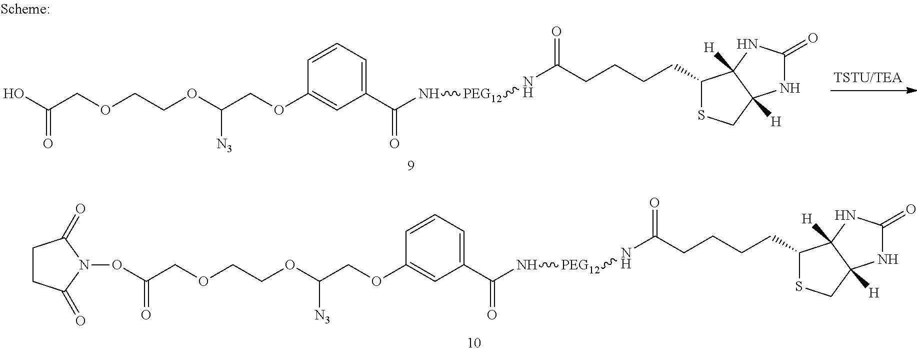

General Procedure for the Preparation of compound 12

To a solution of compound 11, dGTP analogue-amine (3.8 μmol) in NaHCO₃/Na₂CO₃ buffer (0.1 mL, pH 8.7, 0.1 M) was added the DMF solution of biotin-OSu 10 prepared above. The reaction mixture was stirred at room temperature for 3 hours with exclusion of light. LCMS monitoring indicated the formation of desired product. The reaction mixture was directly purified by reverse-phase prep-HPLC (C18, solvent A: 20 nM TEAB in water, solvent B: 20 nM TEAB in MeCN, 5% to 100%) to afford target compound 12, 3′-azidomethyl-dGTP-linker-PEG-biotin.

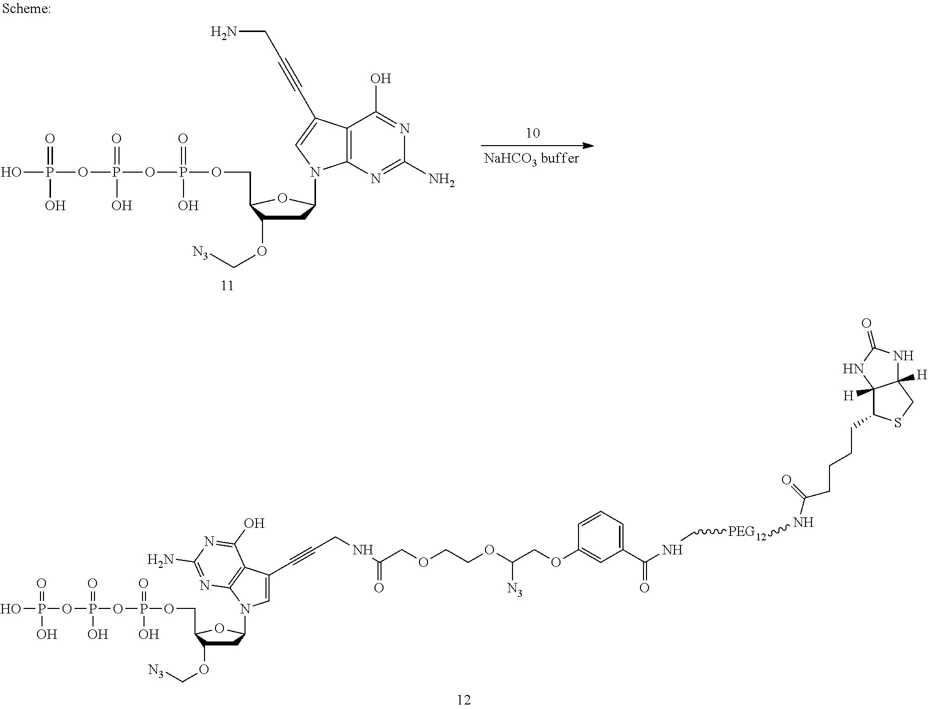

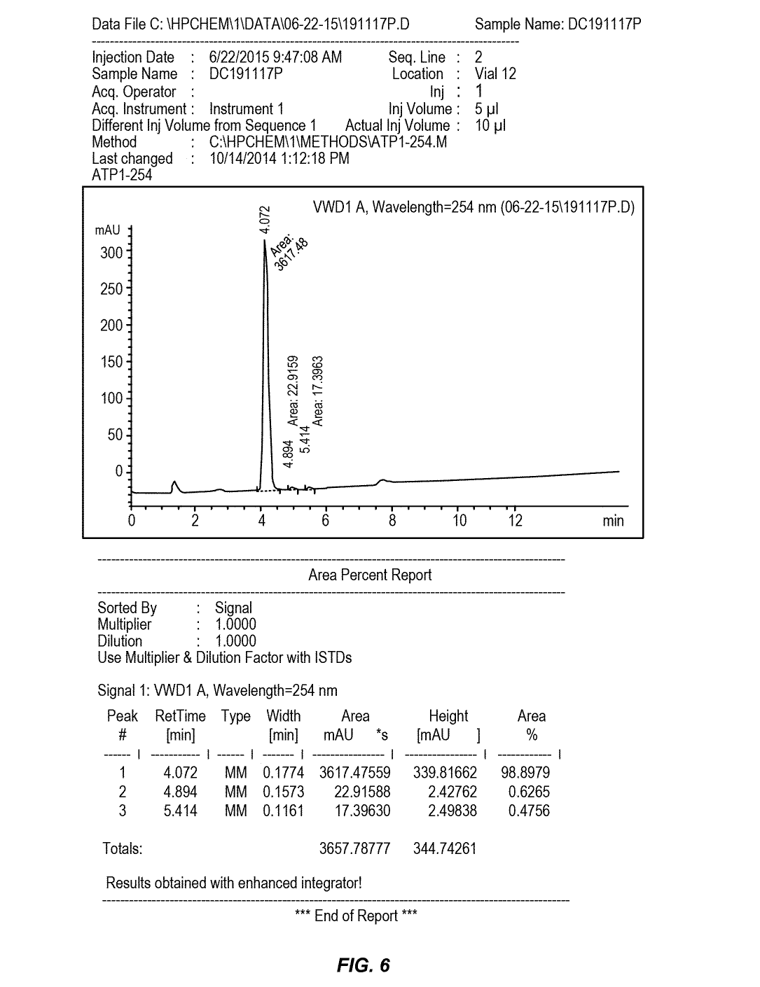

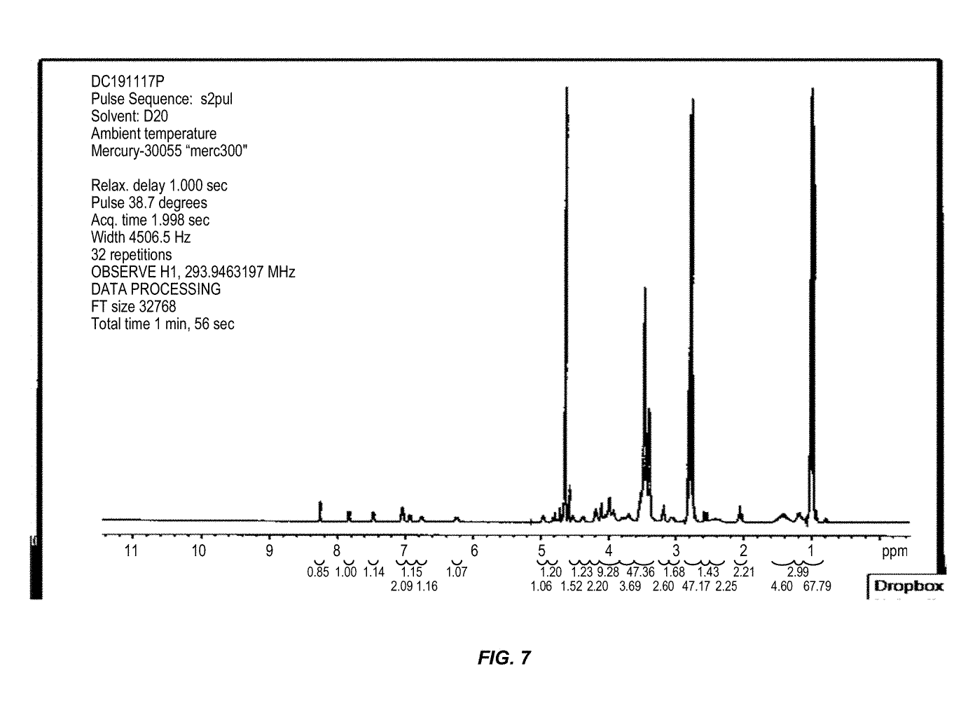

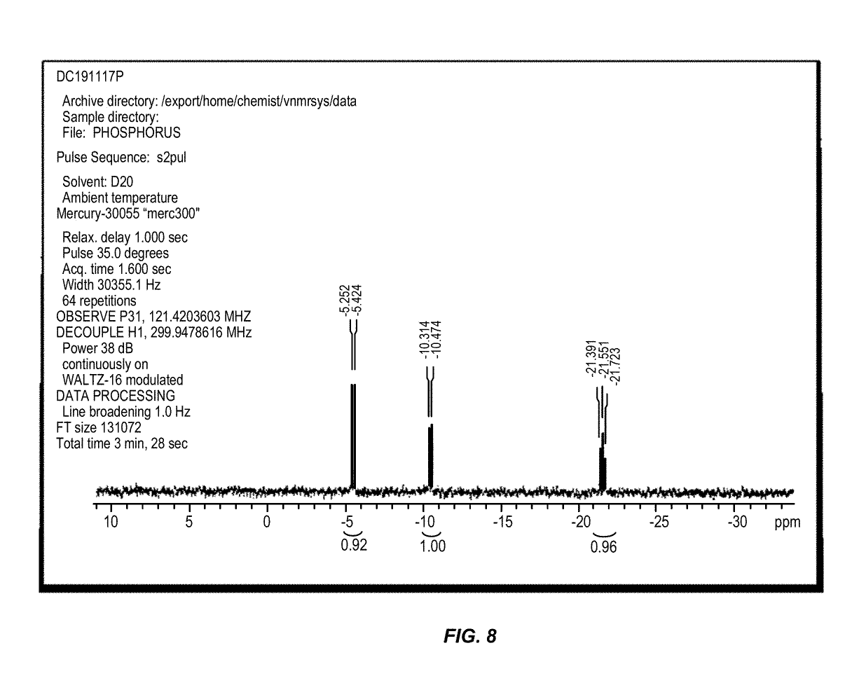

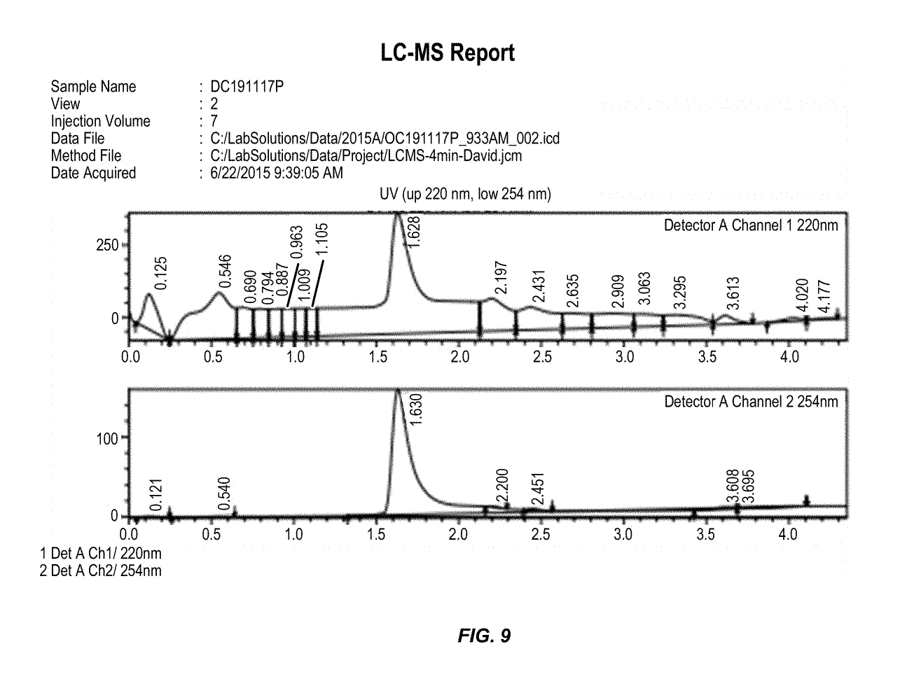

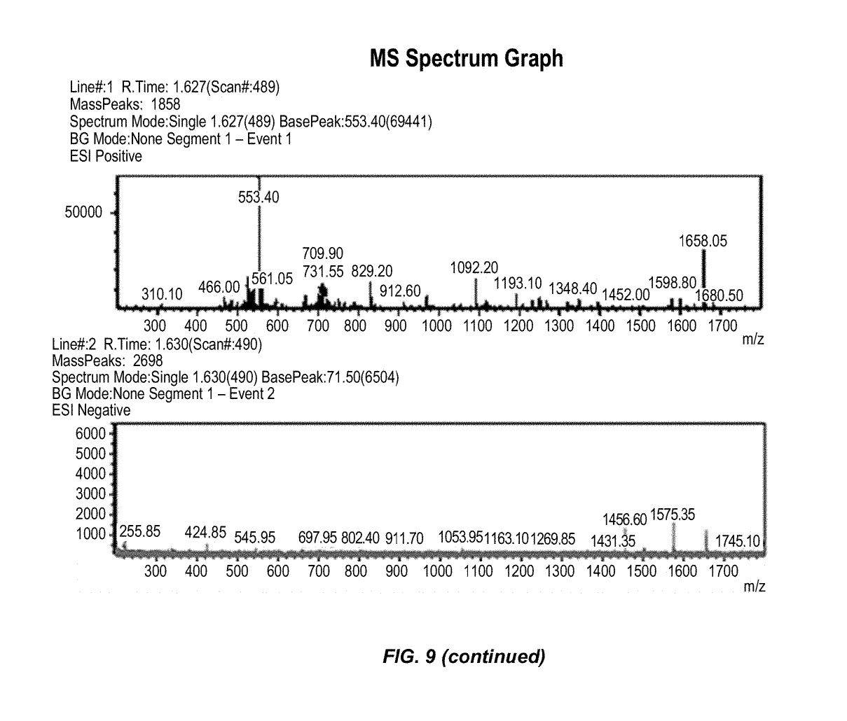

Compound 12 was analyzed by HPLC (FIG. 6), ¹HNMR (FIG. 7), ³¹PNMR (FIG. 8), and LC-MS (FIG. 9), indicating successful production and purification of compound 12.

Alternate affinity tagged nucleoside analogues, such as those shown below are available via similar synthetic routes:

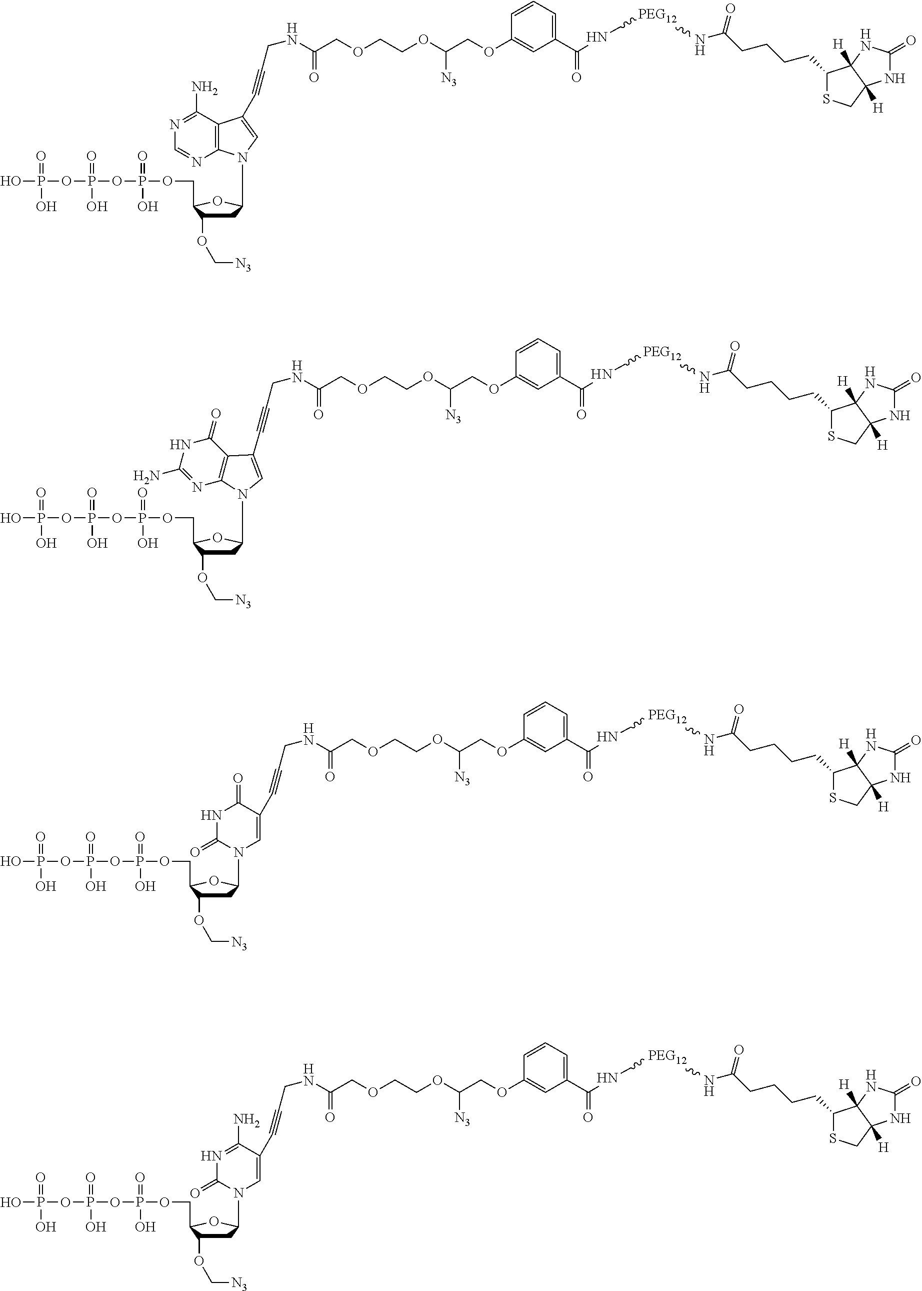

### Example 2

### Sequencing Method

The 3′ reversibly blocked nucleoside analogues containing a nucleobase cleavably linked to a biotin affinity tag, as described herein, were used for sequencing. To perform the sequencing, stock solutions of the following were prepared:

1 mM biotinylated dATP or dCTP analogue in 10 mM Tris-EDTA (TE) buffer pH 8.0;

2 mg/mL FITC-streptavidin in TE buffer 8.0 with 50% glycerol; and

2 mg/mL ifluor 700 streptavidin in TE buffer 8.0 with 50% glycerol.

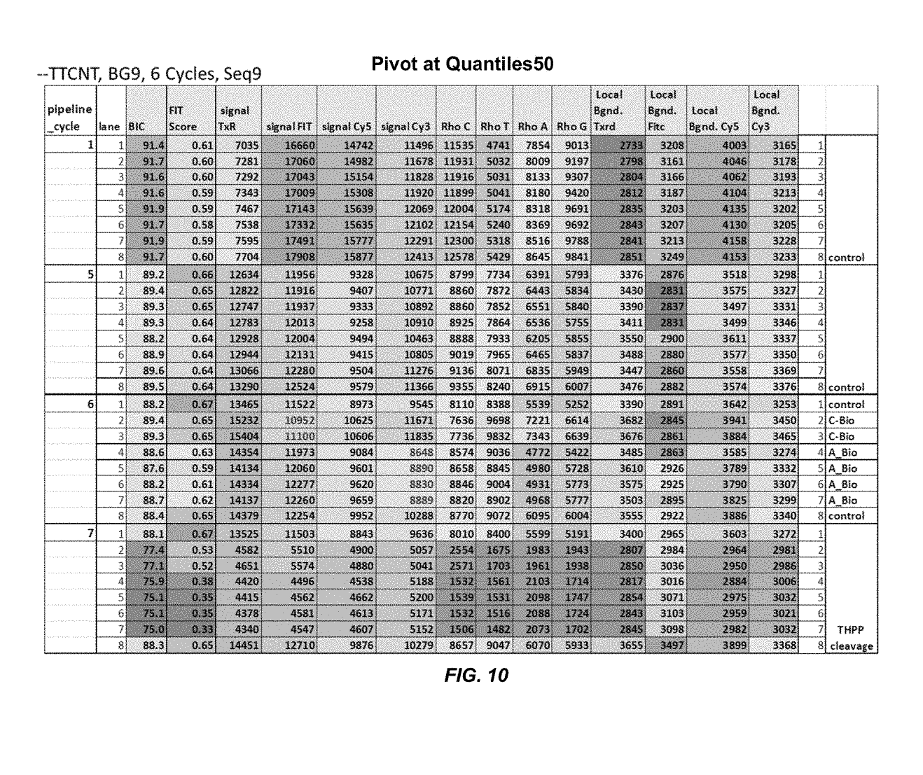

The stock solutions (2 mg/mL) were diluted with a high salt buffer to generate 20 μg/mL labeled streptavidin working solutions containing 500 mM NaCl, 40 mM Tris, pH 8.0. Biotinylated dATP or dCTP analogues were used during one or more cycles of a sequence by synthesis workflow in place of dye-labeled dATP or dCTP to incorporate biotinylated nucleoside analogues onto flow cell, chip, DNB array, or other substrates. A cold-chase was performed with non-labeled dNTPs, and washed with 500 mM NaCl, 50 mM Tris, pH 8.0 wash buffer three times at 30° C. The substrate containing incorporated biotinylated nucleoside analogues was stained with a 20 μg/mL labeled streptavidin working solution for 5 minutes at 30° C. to detectably label incorporated biotinylated nucleoside analogues. The substrate was washed with read buffer containing 50 mM ascorbate, 50 mM Tris, 150 mM NaCl, pH 7.5 three times at 30° C. The substrate was scanned on a sequencer to detect presence or absence of incorporated nucleoside analogues. The biotinylated nucleoside analogue incorporation, cold-chasing, staining, washing, and scanning steps described above were repeated as necessary. Exemplary sequencing data is illustrated in FIG. 10.

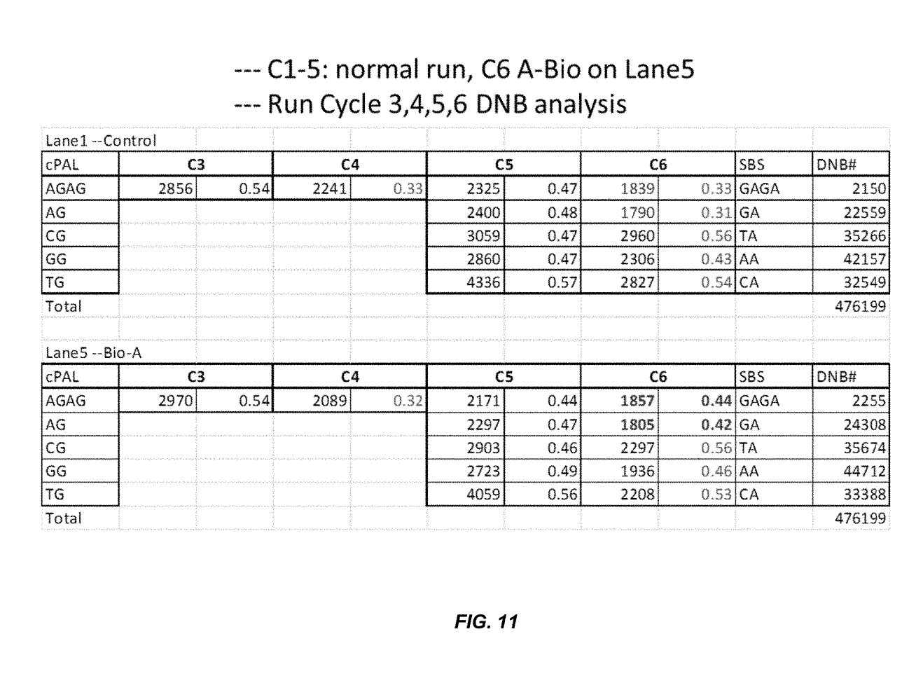

Detection of conventionally labeled nucleoside analogues incorporated into DNA nanoballs illustrates quenching of the fluorescent signal by adjacent G nucleotides. In comparison, detection of affinity tag labeled nucleoside analogues with a fluorescently labeled affinity agent is surprisingly resistant to fluorescence quenching by adjacent G nucleotides (FIG. 11).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A nucleoside analogue of the following formula: 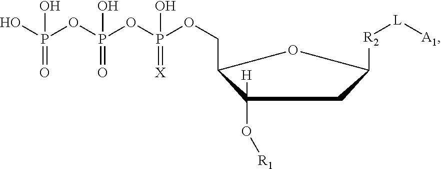
wherein
R₁ is a reversible blocking group selected from the group consisting of azidomethyl, nitrobenzyl, coumarinyl, nitronaphthalenyl, aminoxyl, and carbonyl;
R₂ is a nucleobase;
L is a linker;
A₁ comprises a non-fluorescent affinity tag;
X is selected from the group consisting of O and 5; and
the nucleoside analogue is a substrate for a DNA polymerase.

2. The nucleoside analogue of claim 1, wherein L is a cleavable linker.

3. The nucleoside analogue of claim 2, wherein R₁ and L can be cleaved from the nucleoside analogue under the same conditions.

4. The nucleoside analogue of claim 1, wherein A₁ comprises a non-fluorescent affinity tag selected from the group consisting of nitrilotriacetic acid (NTA) and a peptide comprising at least six contiguous histidine amino acids.

5. The nucleoside analogue of claim 1, wherein A₁ comprises a non-fluorescent affinity tag selected from the group consisting of biotin, vitamin D₃, a non-fluorescent small molecule antigen, and a peptide.

6. The nucleoside analogue of claim 5, wherein the non-fluorescent small molecule antigen is selected from the group consisting of an amphetamine, a barbituate, a benzodiazepine, a cocaine metabolite, a cannabinoid, a cannabinoid metabolite, tetrahydrocannabinol, methadone, an opiate, propoxyphene, phencyclidine, digoxigenin, digoxin, and DNP.

7. The nucleoside analogue of claim 5, wherein the peptide antigen is selected from the group consisting of a His tag, a Myc tag, a Flag tag, an HA tag, a V5 tag, an AviTag, a calmodulin tag, an E tag, an S tag, an SBP tag, a Softag, a Strep tag, a TC tag, a VSV tag, an Xpress tag, glutathione, an isopeptag, and a SpyTag.

8. The nucleoside analogue of claim 5, wherein the nucleoside analogue comprises the following formula: 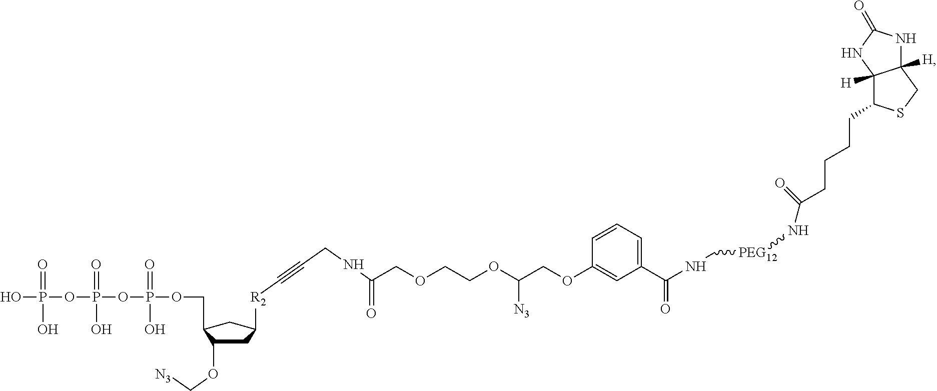
wherein R₂ is the nucleobase.

9. The nucleoside analogue of claim 8, wherein the nucleobase is selected from the group consisting of a 7-substituted 7-deaza adenine analogue, a 7 substituted 7-deaza guanine analogue, a 5-substituted thymine, and a 5-substituted cytosine.

10. A composition comprising
i) a nucleoside analogue of the following formula: 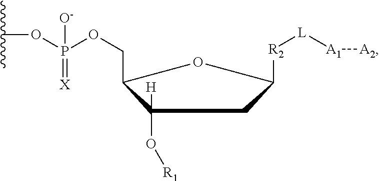
wherein
R₁ is a reversible blocking group selected from the group consisting of azidomethyl, nitrobenzyl, coumarinyl, nitronaphthalenyl, aminoxyl, and carbonyl;
X is selected from the group consisting of O and S;
R₂ is a nucleobase;
L is a linker;
A₁ comprises a fluorescent or non-fluorescent affinity tag; and
A₂ comprises a detectably labeled affinity agent that forms a specific and non-covalent complex with A₁, wherein the nucleoside analogue is covalently linked via the 5′ phosphate or thiophosphate to an oligonucleotide.

11. The composition of claim 10, wherein A₁ comprises a fluorescent dye selected from the group consisting of a fluorone dye, a rhodamine dye, a cyanine dye, a coumarin dye, a phycoerythrin, and an allophycocyanine.

12. A method of sequencing comprising:
i) providing a reaction mixture comprising template nucleic acid, a primer, a polymerase, and a first nucleoside analogue of Formula VII: 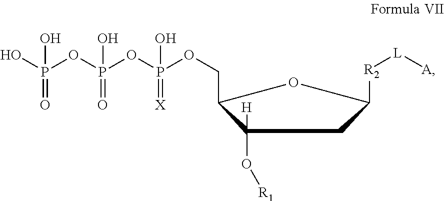
wherein
R₁ is a reversible blocking group selected from the group consisting of azidomethyl, nitrobenzyl, coumarinyl, nitronaphthalenyl, aminoxyl, and carbonyl;
X is selected from the group consisting of 0 and S;
R₂ is a nucleobase;
L is a linker; and
A comprises a non-fluorescent or fluorescent affinity tag;
ii) extending the primer by incorporating the first nucleoside analogue with the polymerase;
iii) contacting the incorporated first nucleoside analogue with a detectably labeled affinity agent that forms a specific and non-covalent complex with A of the incorporated first nucleoside analogue, thereby specifically labeling the incorporated first nucleoside analogue; and
iv) detecting the specifically labeled incorporated first nucleoside analogue.

13. The method of claim 12, wherein the detectably labeled affinity agent is fluorescently labeled, and the detection comprises detecting a fluorescence emission from the fluorescently labeled affinity agent in complex with A of the incorporated first nucleoside analogue.

14. The method of claim 12, wherein the method further comprises:
v) cleaving the reversible blocking group R₁ of the incorporated first nucleoside analogue thereby removing the blocking group from the incorporated first nucleoside analogue;
vi) cleaving the linker L thereby removing the non-fluorescent or fluorescent binding molecule A of the incorporated first nucleoside analogue, or quenching the label of the detectably labeled affinity agent in complex with A of the incorporated first nucleoside analogue;
vii) providing a second nucleoside analogue of Formula VII and a polymerase;
viii) extending the primer by incorporating the second nucleoside analogue with the polymerase;
ix) contacting the incorporated second nucleoside analogue with a detectably labeled affinity agent that forms a specific and non-covalent complex with A of the incorporated second nucleoside analogue, thereby specifically labeling the incorporated second nucleoside analogue; and
x) detecting the specifically labeled incorporated second nucleoside analogue.

15. A method of sequencing comprising:
i) providing a reaction mixture comprising template nucleic acid, a primer, a ligase, and an oligonucleotide comprising a 5′ portion and a 3′ portion comprising a first nucleoside analogue of Formula X: 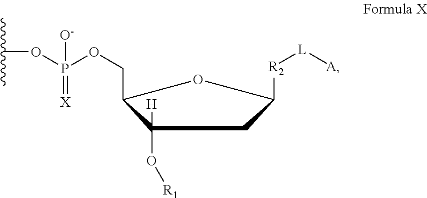
wherein
R₁ is a reversible blocking group selected from the group consisting of azidomethyl, nitrobenzyl, coumarinyl, nitronaphthalenyl, aminoxyl, and carbonyl;
X is selected from the group consisting of O and S;
R₂ is a nucleobase;
L is a linker;
A comprises a non-fluorescent or fluorescent affinity tag; and [symbol]
denotes a 5′ phosphodiester bond between the nucleoside analogue of Formula X and the 5′ portion of the oligonucleotide;
ii) hybridizing the oligonucleotide comprising the first nucleoside analogue of Formula X to the template nucleic acid at a position 3′ of, and adjacent to, the primer;
iii) ligating the hybridized oligonucleotide to the adjacent primer with the ligase, thereby incorporating the first nucleoside analogue of Formula X into the primer;
iv) contacting the incorporated first nucleoside analogue with a detectably labeled affinity agent that forms a specific and non-covalent complex with A of the incorporated first nucleoside analogue, thereby specifically labeling the incorporated first nucleoside analogue; and
v) detecting the specifically labeled incorporated first nucleoside analogue.

16. The method of claim 15, wherein the detectably labeled affinity agent is fluorescently labeled, and the detection comprises detecting a fluorescence emission from the fluorescently labeled affinity agent in complex with A of the incorporated first nucleoside analogue.

17. The method of claim 15, wherein the method further comprises:
vi) cleaving the linker L thereby removing the non-fluorescent or fluorescent binding molecule A of the incorporated first nucleoside analogue, or quenching the label of the detectably labeled affinity agent in complex with A of the incorporated first nucleoside analogue;
vii) cleaving the reversible blocking group R₁ of the hybridized oligonucleotide comprising the first nucleoside analogue of Formula X;
viii) providing a second oligonucleotide, the oligonucleotide comprising a 5′ portion and a 3′ portion, wherein the 3′ portion comprises a second nucleoside analogue of Formula X, and a ligase;
ix) hybridizing the second oligonucleotide comprising the second nucleoside analogue of Formula X to the template nucleic acid at a position 3′ of, and adjacent to, the primer;
x) ligating the hybridized oligonucleotide to the adjacent primer with the ligase, thereby incorporating the second nucleoside analogue of Formula X into the primer;
xi) contacting the incorporated second nucleoside analogue with a detectably labeled affinity agent that forms a specific and non-covalent complex with A of the incorporated second nucleoside analogue, thereby specifically labeling the incorporated second nucleoside analogue; and
xii) detecting the specifically labeled incorporated second nucleoside analogue.
